(19) United States
(12) Patent Application Publication
(10) Pub. No.: US 2012/0114714 A1
(43) Pub. Date: May 10, 2012

(54) METHOD FOR THE TREATMENT AND PREVENTION OF ERECTILE DYSFUNCTION

(76) Inventors: Taehwan KWAK, Yongin-si (KR); Myung-Gyu Park, Gwacheon-Si (KR); Sang-Ku You, Yongin-Si (KR)

(21) Appl. No.: 13/350,419

(22) Filed: Jan. 13, 2012

Related U.S. Application Data
(63) Continuation of application No. 12/515,088, filed on May 15, 2009.

(30) Foreign Application Priority Data
Nov. 27, 2006 (KR) 10-2006-0117685
Jun. 29, 2007 (KR) 10-2007-0065163

(51) Int. Cl. A61K 31/352; A61K 31/382; A61K 31/381; A61K 31/4178; A61K 9/14; A61P 15/10; A61K 31/343; A61K 31/39; B82Y 5/00

(52) U.S. Cl. 424/400; 514/454; 514/468; 514/455; 514/453; 514/437; 514/443; 514/434; 514/397; 977/773; 977/915

(57) ABSTRACT

Disclosed is a pharmaceutical composition for the treatment and/or prevention of erectile dysfunction, comprising (a) a therapeutically effective amount of a compound represented by Formula 1 or 2, and (b) a pharmaceutically acceptable carrier, a diluent or an excipient, or any combination thereof.

This application is a Continuation of co-pending application Ser. No. 12/515,088, filed on May 15, 2009. Application Ser. No. 12/515,088 is the National Phase of PCT International Application No. PCT/KR2007/006013 filed on Nov. 26, 2007, and claims priority under 35 U.S.C. §119(a) to Patent Application No. 10-2006-0117685 filed in Korea on Nov. 27, 2006 and Patent Application No. 10-2007-0065163 filed in Korea on Jun. 29, 2007, all of which are hereby expressly incorporated by reference into the present application.

### FIELD OF THE INVENTION

The present invention relates to a pharmaceutical composition for treating and preventing erectile dysfunction wherein the composition acts as an AMPK activator and thereby exerts superior therapeutic and prophylactic effects on erectile dysfunction.

### BACKGROUND OF THE INVENTION

Erectile dysfunction (also referred to as “ED” or “(male) impotence”) is a medical term that describes the repeated and continuous inability to develop and maintain the penile erection which is an essential process to secure satisfactory sexual intercourse. That is to say, erectile dysfunction refers to a male sexual dysfunction in which sexual intercourse cannot be sustained due to the inability to achieve or maintain the male penile erection. Such erectile dysfunction prevents realization of satisfactory sexual life, causing family troubles and in the severe case, social problems e.g., neurological enervation. For this reason, it is necessary to perform early and continuous treatment of erectile dysfunction.

There are two major causes for the erectile dysfunction: psychogenic and organic causes. The psychogenic cause is attributed to excessive secretion of noradrenaline which results from excessive actions of sympathetic nerves by psychological and mental effects, an increase in the tensity of penile corpus cavernosum smooth muscles, and secretion inhibition of neurotransmitters. The organic erectile dysfunctions are divided into neurogenic, vasculogenic and endocrine erectile dysfunctions according to the cause.

The neurogenic erectile dysfunction is caused by erectile nerve (or fiber) injury which involves insufficient secretion of relaxation neurotransmitters (e.g., acetylcholine and NO) that mediate penile erection at the peripheries of the erectile nerve, and is induced by central nervous diseases including spinal cord injury and multiple sclerosis, or peripheral nervous diseases including diabetes and previous pelvic surgery. Diabetes is the most common cause of the neurogenic erectile dysfunction and induces erectile dysfunction together with peripheral nervous diseases as complications thereof.

The vasculogenic erectile dysfunction is classified into arteriogenic erectile dysfunction due to arterial insufficiency (failure to fill) and venogenic erectile dysfunction due to venous insufficiency (failure to store). The arteriogenic erectile dysfunction is the inability of adequate blood flow to the penile corpus cavernosum which results from a decrease in the inside diameter of erectile arteries or occlusion thereof due to diseases such as arteriosclerosis. The arteriogenic erectile dysfunction may be caused by a variety of factors such as hypertension, diabetes, hyperlipemia and smoking.

The venogenic erectile dysfunction is the inability to completely achieve or maintain penile erection due to failure to store blood within the penile corpus cavernosum which is caused by inadequate closing actions of erectile veins. For example, corpus cavernosum smooth muscles serving as penile erection tissue are injured, or are implicated in fibrous proliferation as sequelae of diabetes, serious arterial diseases or priapism and are thus replaced by fibrous tissues.

The endocrine erectile dysfunction is caused by disorders of hypothalamic-pituitary-gonadal axis (HPTA), hyperprolactinaemia, thyroid diseases, adrenal diseases, disorders of calcium metabolism, and the like. The most common endocrine disease is known to be diabetes.

Recent researches associated with erectile dysfunction are relatively intensely focused on the organic erectile dysfunction. There are several techniques for treating the organic erectile dysfunction available: injection of an erection-inducing agent into the male penile corpus cavernosum; surgical prosthetic implants; endocrine therapy for endocrine erectile dysfunction which involves maintenance of adequate male hormones in blood to promote sexual appetite; and pharmacological therapy. These techniques are generally used in conjunction with psychological therapy to treat the organic erectile dysfunction.

In view of the pharmacological therapy, a drug such as yohimbine is clinically available, and dopamine-based drugs such as apomorphine are being developed. In particular, the development of sildenafil derivatives such as Viagra™ has brought about a great deal of researches on therapeutic agents for erectile dysfunction.

Meanwhile, the erectile tissue of the male genital organs is broadly composed of two parts, i.e., (penile) corpus cavernosum and corpus spongiosum. A number of spiral arteries which extend from cavernous arteries are found in sponge-like cavernous tissue. Relaxation of the penile corpus cavernosum smooth muscle leads to blood flow through the cavernosum arteries to the spiral arteries, thus resulting in penile erection. The expansion of the cavernosum tissue causes the penis to be rigid enough to achieve sexual intercourse.

Accordingly, erection is directly associated with the relaxation of the penile corpus cavernosum smooth muscle so long as blood vessels are not injured themselves and the main factor responsible for mediating this relaxation is nitric oxide (NO) (Burnet AL, 1997). That is to say, as a result of researches on models with respect to aging and diseases such as atherosclerosis, hypertension and diabetes, all of which are closely associated with the erectile dysfunction, there occurred disorders of non-adrenergic, non-cholinergic (NANC) penile corpus cavernosum smooth muscle relaxation due to inadequate NO action, reduction of the activity of nitric oxide synthase (NOS) and disorder of NOS expression in the penis. This result ascertains that the reduction of the physiological activity of NO causes erectile dysfunction.

Nitric oxide (NO) is a gas-phase neurotransmitter which is secreted and diffused from vascular endothelial cells and has been also known as an endothelial derived releasing factor (EDRF). NO is biosynthesized from arginine by NOS enzymes which are activated by stimuli of the parasympathetic nervous system. The NO diffuses into vascular smooth muscles to stimulate an enzyme called guanyl cyclase (GC). Then, the activated GC converts guanosine triphosphate (GPT) into cyclic guanosine monophosphate (cGMP) (Ignarro LJ, 1981). The cGMP generated in accordance with such a mechanism reduces the calcium concentration in cells, leading to relaxation of actin and myosin, thus finally resulting in relaxation of the penile corpus cavernosum smooth muscle. Phosphodiesterase type 5 (PDE 5) is an enzyme which induces the degradation of cGMP to GMP to inhibit the activity of the cGMP, and is known to be found in the male genital organs (Boolell M, et al. 1996).

In this regard, a Sildenafil derivative such as Viagra™ selectively (4,000 folds or more) inhibits the activity of the enzyme (i.e., PDE 5) that hydrolizes the phosphodiester bond to suppress the degradation of cGMP, resulting in maintenance of the concentration of cGMP in the penile corpus cavernosum and relaxation of the smooth muscles, allowing more blood flow to the penis, thereby maintaining the erection.

However, Sildenafil was reported to cause a variety of adverse effects, e.g., headache, flushing, myocardial infarction, cardiac failure, hypotension and cerebral infarction. Thus, there is an increasingly urgent need to develop an efficient substance suitable for use as a safe drug for erectile dysfunction that is capable of substituting for the Sildenafil.

The present inventors found that a specific naphthoquinone compound activates AMP-activated protein kinase (AMPK), thereby exerting potent efficacy for the treatment of erectile dysfunction.

As shown in FIG. 1, AMPK is a phosphorylation enzyme that controls its activity in response to a cells' energy status (i.e., ATP/AMP ratio) that depends upon various factors such as nutritive conditions, motion and stress of the cells. Once activated, AMPK affects a cascade of physiologic events in subsequent mechanisms and thus in vitro and in vivo plays a key role on metabolism of energy sources such as glucose, protein and fat. Accordingly, AMPK activators have drawn a great deal of attention due to their central roles on regulation of metabolic syndrome including obesity, diabetes, metabolic diseases, degenerative diseases and mitochondrial dysfunction-related diseases.

It was reported by Genevieve, et al. (J. Biol. Chem. 279, 20767-74, 2004) that activation of AMPK inhibits the activity of an NOS enzyme that acts as an inflammatory mediator in chronic inflammatory conditions or endotoxin shock, including obesity-related diabetes diseases, thus being efficient for development of medicines having a new mechanism capable of increasing insulin sensitivity. In addition, it was reported that the inhibition of iNOS activity through the AMPK activation may be clinically applied to diseases such as septicemia, multiple sclerosis, myocardial infarction, inflammatory bowel diseases, and pancreatic beta-cell dysfunctions.

It was reported by Zing-ping, et al. (FEBS Letters 443, 285-289, 1999) that AMPK activates endothelial NO synthase through phosphorylation, in the presence of Ca-calmodulin in muscular and cardiac cells of rats. This indicates that AMPK is implicated in cardiac diseases including angina pectoris.

Meanwhile, a variety of pharmaceutical compositions containing a conventional naphthoquinone-based compound as an effective ingredient are known in the art. Of these, β-lapachone is derived from the laphacho tree (Tabebuia avellanedae) which naturally grows in South America, and dunnione and α-dunnione) are derived from the leaves of Streptocarpus dunnii which naturally grows also in South America. These naturally-occurring tricyclic naphthoquinone derivatives have been used not only as anti-cancer medications, but also as medications for the treatment of a Chagas disease known as a representative endemic disease of South America a long time ago, and was also known to exhibit potent efficacies. In particular, pharmacological actions of the naphthoquinone derivatives as anticancer medications have drawn a great deal of attention since they were known to the Western nations. As disclosed in U.S. Pat. No. 5,969,163, a number of anti-cancer drugs employing the tricyclic naphthoquinone derivative are being actually developed by a great deal of research groups.

Despite the various researches, there is no report ascertaining the fact that the tricyclic naphthoquinone derivatives exhibit pharmacologically therapeutic and prophylactic effects on erectile dysfunction.

### SUMMARY OF THE INVENTION

As a result of a variety of extensive and intensive studies and experiments based on the facts described above, the inventors of the present invention have newly ascertained that a given naphthoquinone-based compound induces expression of penile erection-related neurotransmitters and enzymes via activation of AMPK and is thus useful for treating and preventing erectile dysfunction, and have discovered that the compound exerts desirable pharmacological effects, when formulated to be absorbable in a predetermined site. Accordingly, the present invention is finally completed, based on the afore-mentioned findings.

In accordance with an aspect of the present invention, the above and other objects can be accomplished by the provision of a pharmaceutical composition for the treatment and/or prevention of erectile dysfunction, comprising: (a) a therapeutically effective amount of one or more selected from the compounds represented by Formulas 1 and Formula 2 below:

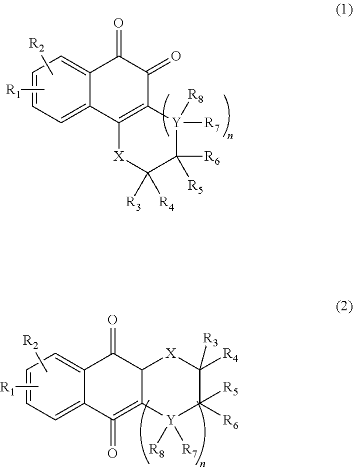

wherein

R₁ and R₂ are each independently hydrogen, halogen, hydroxyl, or C₁-C₆ lower alkyl or alkoxy, or R₁ and R₂ may be taken together to form a substituted or unsubstituted cyclic structure which may be saturated or partially or completely unsaturated;

R₃, R₄, R₅, R₆, R₇ and R₈ are each independently hydrogen, hydroxyl, C₁-C₂₀ alkyl, alkene or alkoxy, or C₄-C₂₀ cycloalkyl, heterocycloalkyl, aryl or heteroaryl, or two substituents of R₃ to R₈ may be taken together to form a cyclic structure which may be saturated or partially or completely unsaturated;

X is selected from the group consisting of C(R)(R′), N(R″), O and S, wherein R, R′ and R″ are each independently hydrogen or C₁-C₆ lower alkyl;

Y is C, S or N, with proviso that R₇ and R₈ are nothing when Y is S, and R₇ is hydrogen or C₁-C₆ lower alkyl and R₈ is nothing when Y is N; and

n is 0 or 1, with proviso that when n is 0, carbon atoms adjacent to n form a cyclic structure via a direct bond; or a pharmaceutically acceptable salt, prodrug, solvate or isomer thereof and

(b) a pharmaceutically acceptable carrier, a diluent or an excipient, or any combination thereof.

As a result of repeated extensive and intensive studies and a variety of experiments, the inventors of the present invention have confirmed that the above naphthoquinone-based compound activates AMPK in cells and tissues and is thus remarkably effective for treating diseases e.g. erectile dysfunction.

More specifically, the pharmaceutical composition of the present invention promotes eNOS activation and cGMP production via activation of AMPK, and thus mediates not only an endothelium-dependent NO-production pathway and a NO-cGMP pathway, but also an endothelium-independent carbon monoxide (CO)-production pathway, thereby inducing relaxation of penile corpus cavernosum smooth muscles. The relaxation elevates blood supply to cavernous arteries and blood flow to spiral arteries, thus resulting in potent therapeutic and prophylactic effects on erectile dysfunction.

Similar to nitric oxide (NO), carbon monoxide (CO) is one of neurotransmitters, and is a vasodilator in vivo endogenously formed in heme by heme oxygenase (HO). HO is classified into heme oxygenase-1 (HO-1) as an inducible isoform and heme oxygenase-2 (HO-2) as a constitutive isoform. HO-2 proteins are only found in the major pelvic ganglion, and HO-2 nerves are found in genitalia, urethra, bladder neck, vas deferens and prostate. The neurotransmitter of the ganglion and nerves has been considered to be CO which is produced from HO. However, the role of CO implicated in penile erection has not yet been sufficiently studied to date.

As a result of repeated experiments associated with the role of CO, the inventors of the present invention have confirmed that the composition according to the present invention activates AMPK, and thereby promotes not only eNOS phosphorylation and eNOS activity by Ca²⁺-calmodulin binding via intracellular calcium release, but also CO production through an endothelium-independent CO pathway, thus being potently effective for inducing relaxation of penile corpus cavernosum smooth muscles. In addition, the inventors have confirmed that the composition according to the present invention is significantly effective for elevating the internal pressure of penile corpus cavernosum smooth muscles in diabetes-inducing rats.

Accordingly, the pharmaceutical composition of the present invention containing the compound represented by Formula 1 or 2 as an active ingredient is useful for treating and preventing erectile dysfunction, in particular, diabetes-related erectile dysfunction.

As used herein, the term “pharmaceutically acceptable salt” means a formulation of a compound that does not cause significant irritation to an organism to which it is administered and does not abrogate the biological activity and properties of the compound. Examples of the pharmaceutical salt may include acid addition salts of the compound with acids capable of forming a non-toxic acid addition salt containing pharmaceutically acceptable anions, for example, inorganic acids such as hydrochloric acid, sulfuric acid, nitric acid, phosphoric acid, hydrobromic acid and hydroiodic acid; organic carbonic acids such as tartaric acid, formic acid, citric acid, acetic acid, trichloroacetic acid, trifluoroacetic acid, gluconic acid, benzoic acid, lactic acid, fumaric acid, maleic acid and salicylic acid; or sulfonic acids such as methanesulfonic acid, ethanesulfonic acid, benzenesulfonic acid and p-toluenesulfonic acid. Specifically, examples of pharmaceutically acceptable carboxylic acid salts include salts with alkali metals or alkaline earth metals such as lithium, sodium, potassium, calcium and magnesium, salts with amino acids such as arginine, lysine and guanidine, salts with organic bases such as dicyclohexylamine, N-methyl-D-glucamine, tris(hydroxymethyl)methylamine, diethanolamine, choline and triethylamine. The compound of the Formula 1 or 2 in accordance with the present invention may be converted into salts thereof, by conventional methods well-known in the art.

As used herein, the term “prodrug” means an agent that is converted into the parent drug in vivo. Prodrugs are often useful because, in some situations, they may be easier to administer than the parent drug. They may, for instance, be bioavailable by oral administration, whereas the parent may be not. The prodrugs may also have improved solubility in pharmaceutical compositions over the parent drug. An example of a prodrug, without limitation, would be a compound of the present invention which is administered as an ester (the “prodrug”) to facilitate transport across a cell membrane where water-solubility is detrimental to mobility, but which then is metabolically hydrolyzed to the carboxylic acid, the active entity, once inside the cell where water solubility is beneficial. A further example of the prodrug might be a short peptide (polyamino acid) bonded to an acidic group, where the peptide is metabolized to reveal the active moiety.

As an example of such prodrug, the pharmaceutical compounds in accordance with the present invention can include a prodrug represented by Formula 1a below as an active material:

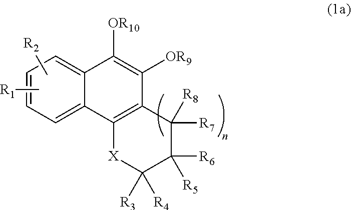

wherein,

R₁, R₂, R₃, R₄, R₅, R₆, R₇, R₈, X and n are as defined in Formula 1;

R₉ and R₁₀ are each independently —SO₃⁻Na⁺ or substituent represented by Formula A below or a salt thereof,

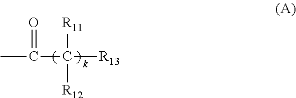

wherein,

R₁₁ and R₁₂ are each independently hydrogen or substituted or unsubstituted C₁-C₂₀ linear alkyl or C₁-C₂₀ branched alkyl

R₁₃ is selected from the group consisting of substituents i) to viii) below:

i) hydrogen;

ii) substituted or unsubstituted C₁-C₂₀ linear alkyl or C₁-C₂₀ branched alkyl;

iii) substituted or unsubstituted amine;

iv) substituted or unsubstituted C₃-C₁₀ cycloalkyl or C₃-C₁₀ heterocycloalkyl;

v) substituted or unsubstituted C₄-C₁₀ aryl or C₄-C₁₀ heteroaryl;

vi) —(CRR′—NR″CO)₁—R₁₄, wherein R, R′ and R″ are each independently hydrogen or substituted or unsubstituted C₁-C₂₀ linear alkyl or C₁-C₂₀ branched alkyl, R₁₄ is selected from the group consisting of hydrogen, substituted or unsubstituted amine, cycloalkyl, heterocycloalkyl, aryl and heteroaryl, 1 is selected from the 1-5;

vii) substituted or unsubstituted carboxyl;

viii) —OSO₃⁻Na⁺;

k is selected from the 0-20, with proviso that when k is 0, R₁₁ and R₁₂ are not anything, and R₁₃ is directly bond to a carbonyl group.

As used herein, the term “solvate” means a compound of the present invention or a salt thereof, which further includes a stoichiometric or non-stoichiometric amount of a solvent bound thereto by non-covalent intermolecular forces. Preferred solvents are volatile, non-toxic, and/or acceptable for administration to humans. Where the solvent is water, the solvate refers to a hydrate.

As used herein, the term “isomer” means a compound of the present invention or a salt thereof that has the same chemical formula or molecular formula but is optically or sterically different therefrom. Unless otherwise specified, the term “compound of Formula 1 or Formula 2” is intended to encompass a compound per se, and a pharmaceutically acceptable salt, prodrug, solvate and isomer thereof.

As used herein, the term “alkyl” refers to an aliphatic hydrocarbon group. The alkyl moiety may be a “saturated alkyl” group, which means that it does not contain any alkene or alkyne moieties. Alternatively, the alkyl moiety may also be an “unsaturated alkyl” moiety, which means that it contains at least one alkene or alkyne moiety. The term “alkene” moiety refers to a group in which at least two carbon atoms form at least one carbon-carbon double bond, and an “alkyne” moiety refers to a group in which at least two carbon atoms form at least one carbon-carbon triple bond. The alkyl moiety, regardless of whether it is substituted or unsubstituted, may be branched, linear or cyclic.

As used herein, the term “heterocycloalkyl” means a carbocyclic group in which one or more ring carbon atoms are substituted with oxygen, nitrogen or sulfur and which includes, for example, but is not limited to furan, thiophene, pyrrole, pyrroline, pyrrolidine, oxazole, thiazole, imidazole, imidazoline, imidazolidine, pyrazole, pyrazoline, pyrazolidine, isothiazole, triazole, thiadiazole, pyran, pyridine, piperidine, morpholine, thiomorpholine, pyridazine, pyrimidine, pyrazine, piperazine and triazine.

As used herein, the term “aryl” refers to an aromatic substituent group which has at least one ring having a conjugated pi (π) electron system and includes both carbocyclic aryl (for example, phenyl) and heterocyclic aryl (for example, pyridine) groups. This term includes monocyclic or fused-ring polycyclic (i.e., rings which share adjacent pairs of carbon atoms) groups.

As used herein, the term “heteroaryl” refers to an aromatic group that contains at least one heterocyclic ring.

Examples of aryl or heteroaryl include, but are not limited to, phenyl, furan, pyran, pyridyl, pyrimidyl and triazyl.

R₁, R₂, R₃, R₄, R₅, R₆, R₇ and R₈ in Formula 1 or Formula 2 in accordance with the present invention may be optionally substituted. When substituted, the substituent group(s) is(are) one or more group(s) individually and independently selected from cycloalkyl, aryl, heteroaryl, heteroalicyclic, hydroxy, alkoxy, aryloxy, mercapto, alkylthio, arylthio, cyano, halogen, carbonyl, thiocarbonyl, O-carbamyl, N carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino including mono and di substituted amino, and protected derivatives thereof. Further, substituents of R₁₁, R₁₂ and R₁₃ in the Formula 1a may be also substituted as defined in above, and when substituted, they can be substituted as the substituents mentioned above.

Among compounds of Formula 1, preferred are compounds of Formulas 3 and 4 below.

Compounds of Formula 3 are compounds wherein n is 0 and adjacent carbon atoms form a cyclic structure (furan ring) via a direct bond therebetween and are often referred to as “furan compounds” or “furan-o-naphthoquinone derivatives” hereinafter.

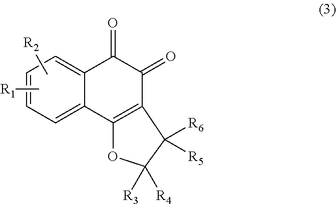

Compounds of Formula 4 are compounds wherein n is 1 and are often referred to as “pyran compounds” or “pyrano-o-naphthoquinone” hereinafter.

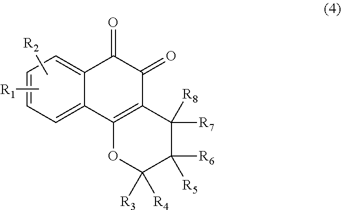

In Formula 1, each of R₁ and R₂ is particularly preferably hydrogen.

Among the furan compounds of Formula 3, particularly preferred are compounds of Formula 3a wherein R₁, R₂ and R₄ are hydrogen, or compounds of Formula 3b wherein R₁, R₂ and R₆ are hydrogen.

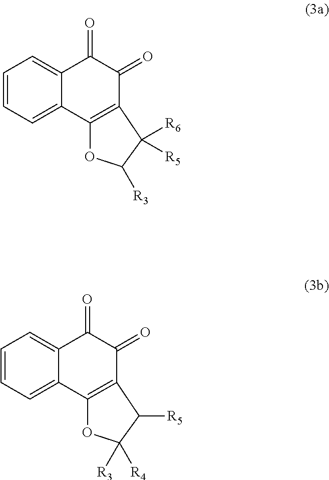

Further, among the pyran compounds of Formula 4, particularly preferred are compounds of Formula 4a wherein R₁, R₂, R₅, R₆, R₇ and R₈ are respectively hydrogen, or compounds of Formula 4b or Formula 4c wherein R₁ and R₂ are taken together to form a cyclic structure which is substituted or unsubstituted.

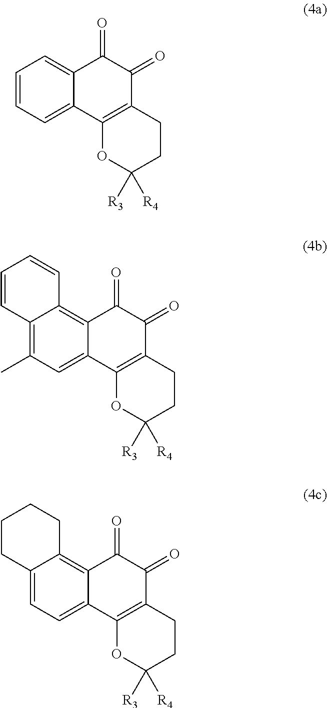

Among the compounds of Formula 2, preferred are compounds of Formula 2a and 2b, but are not limited thereto.

Compounds of Formula 2a below are compounds in which n is 0 and adjacent carbon atoms form a cyclic structure via a direct bond therebetween, and Y is C.

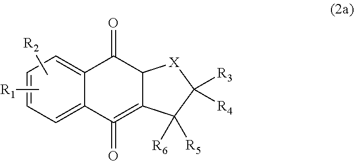

Compounds of Formula 2b below are compounds wherein n is 1 and Y is C in Formula 2.

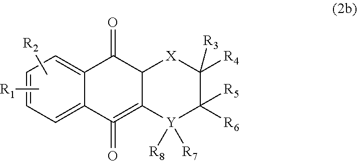

In the formula 2a or 2b, R₁, R₂, R₃, R₄, R₅, R₆, R₇, R₈ and X are as defined in Formula 2.

The term “pharmaceutical composition” as used herein means a mixture of a compound of Formula 1 or Formula 2 with other chemical components, such as diluents or carriers. The pharmaceutical composition facilitates administration of the compound to an organism. Various techniques of administering a compound are known in the art and include, but are not limited to oral, injection, aerosol, parenteral and topical administrations. Pharmaceutical compositions can also be obtained by reacting compounds of interest with acids such as hydrochloric acid, hydrobromic acid, sulfuric acid, nitric acid, phosphoric acid, methanesulfonic acid, p-toluenesulfonic acid, salicylic acid and the like. In the present invention, effective substance which have to exert therapeutic effects on prevention or treatment of-erectile dysfunction comprises compounds represented by the above-mentioned Formulas and is often referred to as “active ingredient” hereinafter.

The term “therapeutically effective amount” means an amount of an active ingredient that is effective to relieve or reduce to some extent one or more of the symptoms of the disease in need of treatment, or to retard initiation of clinical markers or symptoms of a disease in need of prevention, when the compound is administered. Thus, a therapeutically effective amount refers to an amount of the active ingredient which exhibit effects of (i) reversing the rate of progress of a disease; (ii) inhibiting to some extent further progress of the disease; and/or, (iii) relieving to some extent (or, preferably, eliminating) one or more symptoms associated with the disease. The therapeutically effective amount may be empirically determined by experimenting with the compounds concerned in known in vivo and in vitro model systems for a disease in need of treatment.

In the pharmaceutical composition in accordance with the present invention, compounds of Formula 1 or Formula 2 which are active materials, as will be illustrated hereinafter, can be prepared by conventional methods known in the art and/or various processes which are based upon the general technologies and practices in the organic chemistry synthesis field. The preparation processes described below are only exemplary ones and other processes can also be employed. As such, the scope of the instant invention is not limited to the following processes.

### Preparation Method 1

### Synthesis of Active Materials by Acid-Catalyzed Cyclization

Tricyclic naphthoquinone (pyrano-o-naphthoquinone and furano-o-naphthoquinone) derivatives having a relatively simple chemical structure are generally synthesized in a relatively high yield via cyclization using sulfuric acid as a catalyst, Based on this process, a variety of compounds of Formula 1 can be synthesized.

More specifically, the above synthesis process may be summarized as follows.

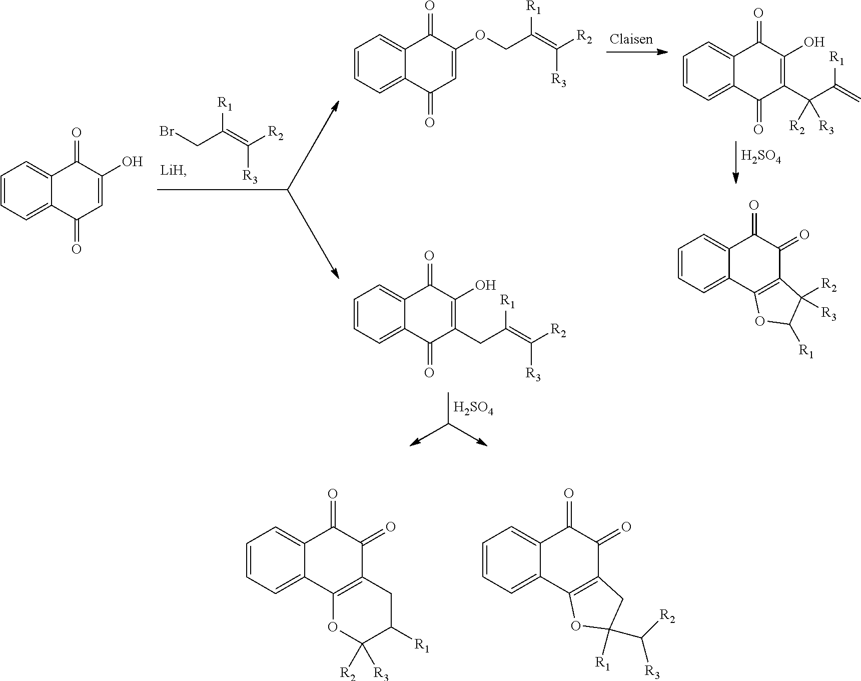

That is, when 2-hydroxy-1,4-naphthoquinone is reacted with various allylic bromides or equivalents thereof in the presence of a base, a C-alkylation product and an O-alkylation product are concurrently obtained. It is also possible to synthesize either of two derivatives only depending upon reaction conditions. Since O-alkylated derivative is converted into another type of C-alkylated derivative through Claisen Rearrangement by refluxing the O-alkylated derivative using a solvent such as toluene or xylene, it is possible to obtain various types of 3-substituted-2-hydroxy-1,4-naphthoquinone derivatives. The various types of C-alkylated derivatives thus obtained may be subjected to cyclization using sulfuric acid as a catalyst, thereby being capable of synthesizing pyrano-o-naphthoquinone or furano-o-naphthoquinone derivatives among compounds of Formula 1.

### Preparation Method 2

### Diels-Alder Reaction Using 3-Methylene-1,2,4-[3H]naphthalenetrione

As taught by V. Nair et al, Tetrahedron Lett. 42 (2001), 4549-4551, it is reported that a variety of pyrano-o-naphthoquinone derivatives can be relatively easily synthesized by subjecting 3-methylene-1,2,4-[3H]naphthalenetrione, produced upon heating 2-hydroxy-1,4-naphthoquinone and formaldehyde together, to Diels-Alder reaction with various olefin compounds. This method is advantageous in that various forms of pyrano-o-naphtho-quinone derivatives can be synthesized in a relatively simplified manner, as compared to induction of cyclization using sulfuric acid as a catalyst

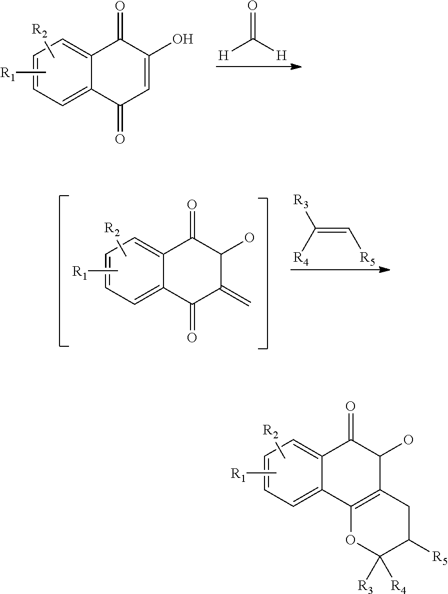

### Preparation Method 3

### Haloakylation and Cyclization by Radical Reaction

The same method used in synthesis of Cryptotanshinone and 15,16-dihydro-tanshinone can also be conveniently employed for synthesis of furan-o-naphthoquinone derivatives. That is, as taught by A. C. Baillie et al (J. Chem. Soc. (C) 1968, 48-52), 2-haloethyl or 3-haloethyl radical chemical species, derived from 3-halopropanoic acid or 4-halobutanoic acid derivative, can be reacted with 2-hydroxy-1,4-naphthoquinone to thereby synthesize 3-(2-haloethyl or 3-halopropyl)-2-hydroxy-1,4-naphthoquinone which is then subjected to cyclization under suitable acidic catalyst conditions to synthesize various pyrano-o-naphthoquinone or furano-o-naphthoquinone derivatives.

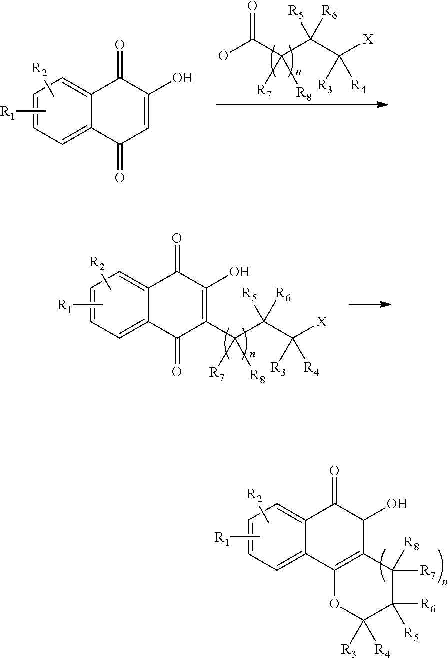

### Preparation Method 4

### Cyclization of 4,5-Benzofurandione by Diels-Alder Reaction

Mother method used in synthesis of Cryptotanshinone and 15,16-dihydro-tanshinone may be a method taught by J. K. Snyder et al (Tetrahedron Letters 28 (1987), 3427-3430). According to this method, furano-o-naphthoquinone derivatives can be synthesized by cycloaddition via Diels-Alder reaction between 4,5-benzofurandione derivatives and various diene derivatives.

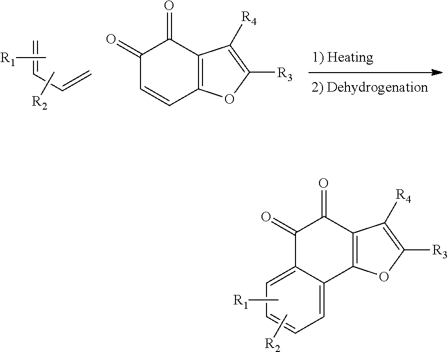

In addition, based on the above-mentioned preparation methods, various derivatives may be synthesized using relevant synthesis methods, depending upon kinds of substituents. Specific examples of derivatives thus synthesized and methods are exemplified in Table 1 below. Specific preparation methods will be described in the following Example.

TABLE 1

| | | | Molec- | Prepa- |
|---|---|---|---|---|
| | | | ular | ration |
| No. | Chemical structure | Formula | weight | method |
| 1 | 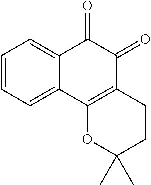 | C₁₅H₁₄O₃ | 242.27 | Method 1 |
| 2 | 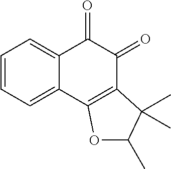 | C₁₅H₁₄O₃ | 242.27 | Method 1 |
| 3 | 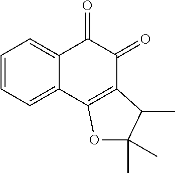 | C₁₅H₁₄O₃ | 242.27 | Method 1 |
| 4 | 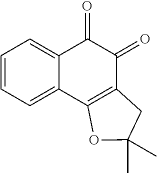 | C₁₄H₁₂O₃ | 228.24 | Method 1 |
| 5 | 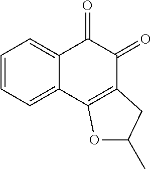 | C₁₃H₁₀O₃ | 214.22 | Method 1 |
| 6 | 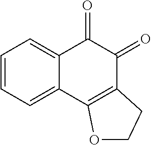 | C₁₂H₈O₃ | 200.19 | Method 2 |
| 7 | 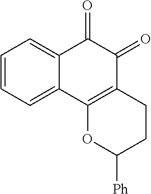 | C₁₉H₁₄O₃ | 290.31 | Method 1 |
| 8 | 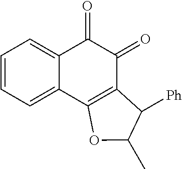 | C₁₉H₁₄O₃ | 290.31 | Method 1 |
| 9 | 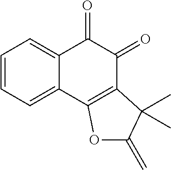 | C₁₅H₁₂O₃ | 240.25 | Method 1 |
| 10 | 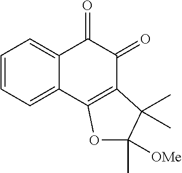 | C₁₆H₁₆O₄ | 272.30 | Method 1 |
| 11 | 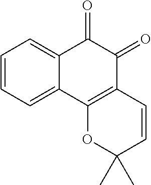 | C₁₅H₁₂O₃ | 240.25 | Method 1 |
| 12 | 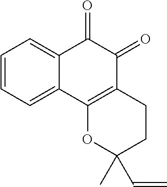 | C₁₆H₁₄O₃ | 254.28 | Method 2 |
| 13 | 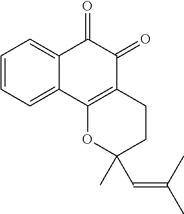 | C₁₈H₁₈O₃ | 282.33 | Method 2 |
| 14 | 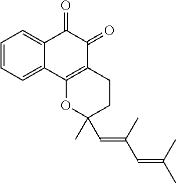 | C₂₁H₂₂O₃ | 322.40 | Method 2 |
| 15 | 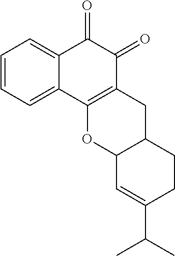 | C₂₁H₂₂O₃ | 322.40 | Method 2 |
| 16 | 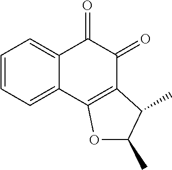 | C₁₄H₁₂O₃ | 228.24 | Method 1 |
| 17 | 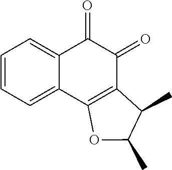 | C₁₄H₁₂O₃ | 228.24 | Method 1 |
| 18 | 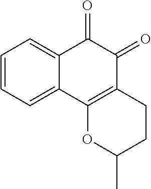 | C₁₄H₁₂O₃ | 228.24 | Method 1 |
| 19 | 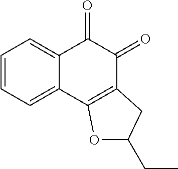 | C₁₄H₁₂O₃ | 228.24 | Method 1 |
| 20 | 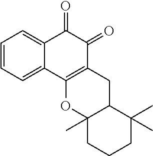 | C₂₀H₂₂O₃ | 310.39 | Method 1 |
| 21 | 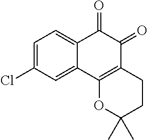 | C₁₅H₁₃ClO₃ | 276.71 | Method 1 |
| 22 | 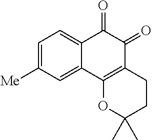 | C₁₆H₁₆O₃ | 256.30 | Method 1 |
| 23 | 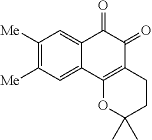 | C₁₇H₁₈O₅ | 302.32 | Method 1 |
| 24 | 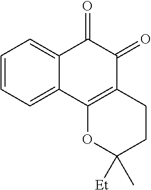 | C₁₆H₁₆O₃ | 256.30 | Method 1 |
| 25 | 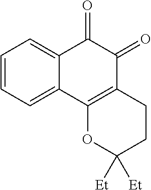 | C₁₇H₁₈O₃ | 270.32 | Method 1 |
| 26 | 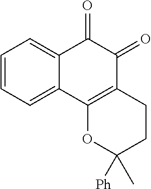 | C₂₀H₁₆O₃ | 304.34 | Method 1 |
| 27 | 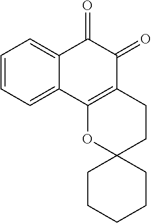 | C₁₈H₁₈O₃ | 282.33 | Method 1 |
| 28 | 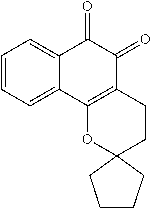 | C₁₇H₁₆O₃ | 268.31 | Method 1 |
| 29 | 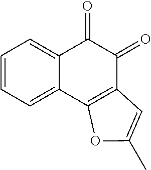 | C₁₃H₈O₃ | 212.20 | Method 1 |
| 30 | 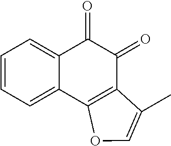 | C₁₃H₈O₃ | 212.20 | Method 4 |
| 31 | 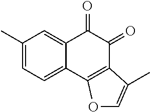 | C₁₄H₁₀O₃ | 226.23 | Method 4 |
| 32 | 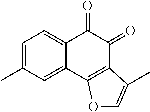 | C₁₄H₁₀O₃ | 226.23 | Method 4 |
| 33 | 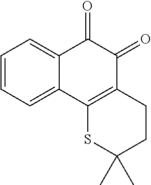 | C₁₅H₁₄O₂S | 258.34 | Method 1 |
| 34 | 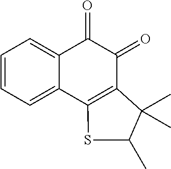 | C₁₅H₁₄O₂S | 258.34 | Method 1 |
| 35 | 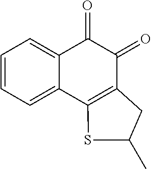 | C₁₃H₁₀O₂S | 230.28 | Method 1 |
| 36 | 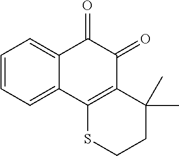 | C₁₅H₁₄O₂S | 258.34 | Method 2 |
| 37 | 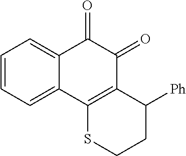 | C₁₉H₁₄O₂S | 306.38 | Method 2 |
| 38 | 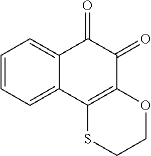 | C₁₂H₈O₃S | 232.26 | Method 3 |
| 39 | 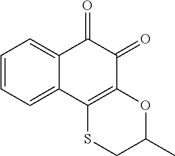 | C₁₃H₁₀O₃S | 246.28 | Method 3 |
| 40 | 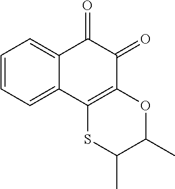 | C₁₄H₁₂O₃S | 260.31 | Method 3 |
| 41 | 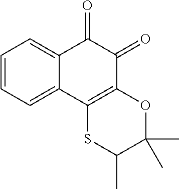 | C₁₅H₁₄O₃S | 274.34 | Method 3 |
| 42 | 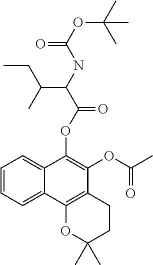 | C₂₈H₃₇O₇N | 502.22 | — |
| 43 | 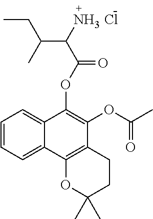 | C₂₃H₃₀O₅NCl | 940.32 | — |
| 44 | 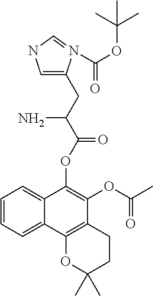 | C₂₈H₃₃O₇N₃ | 526.22 | — |
| 45 | 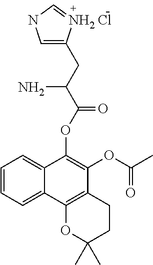 | C₂₃H₂₆O₅N₃Cl | 988.32 | — |
| 46 | 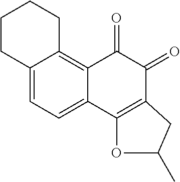 | C₁₇H₁₆O₃ | 268.31 | — |
| 47 | 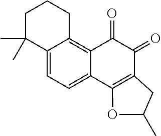 | C₁₉H₂₀O₃ | 296.36 | — |
| 48 | 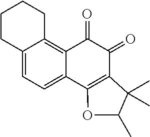 | C₁₉H₂₀O₃ | 296.36 | — |
| 49 | 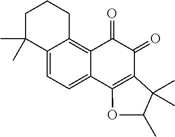 | C₂₁H₂₄O₃ | 324.41 | — |
| 50 | 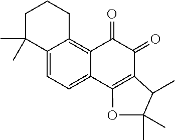 | C₂₁H₂₄O₃ | 324.41 | — |
| 51 | 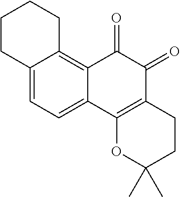 | C₁₉H₂₀O₃ | 296.36 | — |
| 52 | 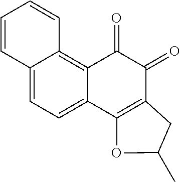 | C₁₇H₁₂O₃ | 264.28 | — |
| 53 | 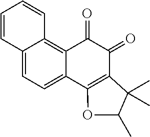 | C₁₉H₁₆O₃ | 292.33 | — |
| 54 | 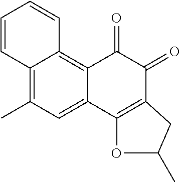 | C₁₈H₁₄O₃ | 278.30 | — |
| 55 | 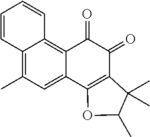 | C₂₀H₁₈O₃ | 306.36 | — |
| 56 | 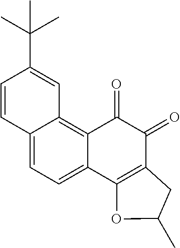 | C₂₁H₂₀O₃ | 320.38 | — |
| 57 | 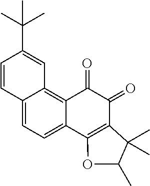 | C₂₃H₂₄O₃ | 348.43 | — |
| 58 | 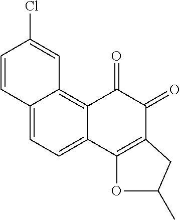 | C₁₇H₁₁ClO₃ | 298.72 | — |
| 59 | 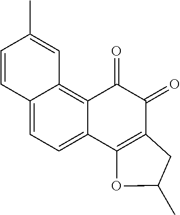 | C₁₈H₁₄O₃ | 278.30 | — |
| 60 | 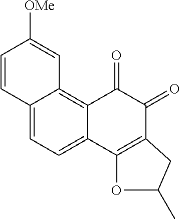 | C₁₈H₁₄O₄ | 294.30 | — |
| 61 | 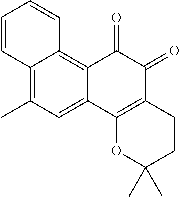 | C₂₀H₁₈O₃ | 306.36 | — |
| 62 | 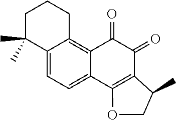 | C₁₈H₁₈O₃ | 282.33 | — |
| 63 | 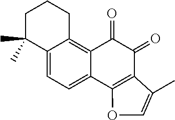 | C₁₈H₁₆O₃ | 280.33 | — |
| 64 | 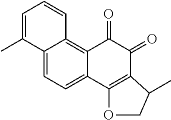 | C₁₈H₁₄O₃ | 278.33 | — |
| 65 | 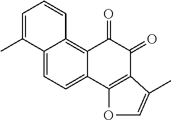 | C₁₈H₁₂O₃ | 276.33 | — |

The pharmaceutical composition of the present invention may be manufactured in a manner that is itself known, e.g., by means of conventional mixing, dissolving, granulating, dragee-making, levigating, emulsifying, encapsulating, entrapping or lyophilizing processes.

Therefore, pharmaceutical compositions for use in accordance with the present invention may be additionally comprised of a pharmaceutically acceptable carrier, a diluent or an excipient, or any combination thereof. That may be formulated in a conventional manner using one or more pharmaceutically acceptable carriers comprising excipients and auxiliaries which facilitate processing of the active compounds into preparations which can be used pharmaceutically. The pharmaceutical composition facilitates administration of the compound to an organism.

The term “carrier” means a chemical compound that facilitates the incorporation of a compound into cells or tissues. For example, dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffer solution is phosphate buffered saline (PBS) because it mimics the ionic strength conditions of human body fluid. Since buffer salts can control the pH of a solution at low concentrations, a buffer diluent rarely modifies the biological activity of a compound.

The compounds described herein may be administered to a human patient per se, or in the form of pharmaceutical compositions in which they are mixed with other active ingredients, as in combination therapy, or suitable carriers or excipient(s). Proper formulation is dependent upon the route of administration chosen. Techniques for formulation and administration of the compounds may be found in “Remington's Pharmaceutical Sciences,” Mack Publishing Co., Easton, Pa., 18th edition, 1990.

Various techniques relating to pharmaceutical formulation for administering an active ingredient into the body are known in the art and include, but are not limited to oral, injection, aerosol, parenteral and topical administrations. If necessary, they can also be obtained by reacting compounds of interest with acids such as hydrochloric acid, hydrobromic acid, sulfuric acid, nitric acid, phosphoric acid, methanesulfonic acid, p-toluenesulfonic acid, salicylic acid and the like.

Pharmaceutical formulation may be carried out by conventional methods known in the art and, Preferably, the pharmaceutical formulation may be oral, external, transdermal, transmucosal and an injection formulation, and particularly preferred is oral formulation.

The pharmaceutical compounds in accordance with the present invention, may be particularly preferably an oral pharmaceutical composition which is prepared into an intestine-targeted formulation.

Generally, an oral pharmaceutical composition passes through the stomach upon oral administration, is largely absorbed by the small intestine and then diffused into all the tissues of the body, thereby exerting therapeutic effects on the target tissues.

In this connection, the oral pharmaceutical composition according to the present invention enhances bioabsorption and bioavailability of a compound of Formula 1 or Formula 2 active ingredient via intestine-targeted formulation of the active ingredient. More specifically, when the active ingredient in the pharmaceutical composition according to the present invention is primarily absorbed in the stomach, and upper parts of the small intestine, the active ingredient absorbed into the body directly undergoes liver metabolism which is then accompanied by substantial degradation of the active ingredient, so it is impossible to exert a desired level of therapeutic effects. On the other hand, it is expected that when the active ingredient is largely absorbed around and downstream of the lower small intestine, the absorbed active ingredient migrates via lymph vessels to the target tissues to thereby exert high therapeutic effects.

Further, as it is constructed in such a way that the pharmaceutical composition according to the present invention targets up to the colon which is a final destination of the digestion process, it is possible to increase the in vivo retention time of the drug and it is also possible to minimize decomposition of the drug which may take place due to the body metabolism upon administration of the drug into the body. As a result, it is possible to improve pharmacokinetic properties of the drug, to significantly lower a critical effective dose of the active ingredient necessary for the treatment of the disease, and to obtain desired therapeutic effects even with administration of a trace amount of the active ingredient. Further, in the oral pharmaceutical composition, it is also possible to minimize the absorption variation of the drug by reducing the between- and within-individual variation of the bioavailability which may result from intragastric pH changes and dietary uptake patterns.

Therefore, the intestine-targeted formulation according to the present invention is configured such that the active ingredient is largely absorbed in the small and large intestines, more preferably in the jejunum, and the ileum and colon corresponding to the lower small intestine, particularly preferably in the ileum or colon.

The intestine-targeted formulation may be designed by taking advantage of numerous physiological parameters of the digestive tract, through a variety of methods. In one preferred embodiment of the present invention, the intestine-targeted formulation may be prepared by (1) a formulation method based on a pH-sensitive polymer, (2) a formulation method based on a biodegradable polymer which is decomposable by an intestine-specific bacterial enzyme, (3) a formulation method based on a biodegradable matrix which is decomposable by an intestine-specific bacterial enzyme, or (4) a formulation method which allows release of a drug after a given lag time, and any combination thereof.

Specifically, the intestine-targeted formulation (1) using the pH-sensitive polymer is a drug delivery system which is based on pH changes of the digestive tract. The pH of the stomach is in a range of 1 to 3, whereas the pH of the small and large intestines has a value of 7 or higher, as compared to that of the stomach. Based on this fact, the pH-sensitive polymer may be used in order to ensure that the pharmaceutical composition reaches the lower intestinal parts without being affected by pH fluctuations of the digestive tract Examples of the pH-sensitive polymer may include, but are not limited to, at least one selected from the group consisting of methacrylic acid-ethyl acrylate copolymer (Eudragit: Registered Trademark of Rohm Pharma GmbH), hydroxypropylmethyl cellulose phthalate (HPMCP) and a mixture thereof.

Preferably, the pH-sensitive polymer may be added by a coating process. For example, addition of the polymer may be carried out by mixing the polymer in a solvent to form an aqueous coating suspension, spraying the resulting coating suspension to form a film coating, and drying the film coating.

The intestine-targeted formulation (2) using the biodegradable polymer which is decomposable by the intestine-specific bacterial enzyme is based on the utilization of a degradative ability of a specific enzyme that can be produced by enteric bacteria. Examples of the specific enzyme may include azoreductase, bacterial hydrolase glycosidase, esterase, polysaccharidase, and the like.

When it is desired to design the intestine-targeted formulation using azoreductase as a target, the biodegradable polymer may be a polymer containing an azoaromatic linkage, for example, a copolymer of styrene and hydroxyethylmethacrylate (HEMA). When the polymer is added to the formulation containing the active ingredient, the active ingredient may be liberated into the intestine by reduction of an azo group of the polymer via the action of the azoreductase which is specifically secreted by enteric bacteria, for example, Bacteroides fragilis and Eubacterium limosum.

When it is desired to design the intestine-targeted formulation using glycosidase, esterase, or polysaccharidase as a target, the biodegradable polymer may be a naturally-occurring polysaccharide or a substituted derivative thereof. For example, the biodegradable polymer may be at least one selected from the group consisting of dextran ester, pectin, amylose, ethyl cellulose and a pharmaceutically acceptable salt thereof. When the polymer is added to the active ingredient, the active ingredient may be liberated into the intestine by hydrolysis of the polymer via the action of each enzyme which is specifically secreted by enteric bacteria, for example, Bifidobacteria and Bacteroides spp. These polymers are natural materials, and have an advantage of low risk of in vivo toxicity.

The intestine-targeted formulation (3) using the biodegradable matrix which is decomposable by an intestine-specific bacterial enzyme may be a form in which the biodegradable polymers are cross-linked to each other and are added to the active ingredient or the active ingredient-containing formulation. Examples of the biodegradable polymer may include naturally-occurring polymers such as chondroitin sulfate, guar gum, chitosan, pectin, and the like. The degree of drug release may vary depending upon the degree of cross-linking of the matrix-constituting polymer.

In addition to the naturally-occurring polymers, the biodegradable matrix may be a synthetic hydrogel based on N-substituted acrylamide. For example, there may be used a hydrogel synthesized by cross-linking of N-tert-butylacryl amide with acrylic acid or copolymerization of 2-hydroxyethyl methacrylate and 4-methacryloyloxyazobenzene, as the matrix. The cross-linking may be, for example an azo linkage as mentioned above, and the formulation may be a form where the density of cross-linking is maintained to provide the optimal conditions for intestinal drug delivery and the linkage is degraded to interact with the intestinal mucous membrane when the drug is delivered to the intestine.

Further, the intestine-targeted formulation (4) with time-course release of the drug after a lag time is a drug delivery system utilizing a mechanism that is allowed to release the active ingredient after a predetermined time irrespective of pH changes. In order to achieve enteric release of the active drug, the formulation should be resistant to the gastric pH environment, and should be in a silent phase for 5 to 6 hours corresponding to a time period taken for delivery of the drug from the body to the intestine, prior to release of the active ingredient into the intestine. The time-specific delayed-release formulation may be prepared by addition of the hydrogel prepared from copolymerization of polyethylene oxide with polyurethane.

Specifically, the delayed-release formulation may have a configuration in which the formulation absorbs water and then swells while it stays within the stomach and the upper digestive tract of the small intestine, upon addition of a hydrogel having the above-mentioned composition after applying the drug to an insoluble polymer, and then migrates to the lower part of the small intestine which is the lower digestive tract and liberates the drug, and the lag time of drug is determined depending upon a length of the hydrogel.

As another example of the polymer, ethyl cellulose (EC) may be used in the delayed-release dosage formulation. EC is an insoluble polymer, and may serve as a factor to delay a drug release time, in response to swelling of a swelling medium due to water penetration or changes in the internal pressure of the intestines due to a peristaltic motion. The lag time may be controlled by the thickness of EC. As an additional example, hydroxypropylmethyl cellulose (HPMC) may also be used as a retarding agent that allows drug release after a given period of time by thickness control of the polymer, and may have a lag time of 5 to 10 hours.

In the oral pharmaceutical composition according to the present invention, the active ingredient may have a crystalline structure with a high degree of crystallinity, or a crystalline structure with a low degree of crystallinity. Preferably, the active ingredient is composed of the crystalline structure with a low crystallinity degree, which can solve the problems associated with poor solubility of the compound of Formula 1 or 2 and increase the dissolution rate and in vivo absorption rate thereof.

As used herein, the term “degree of crystallinity” is defined as the weight fraction of the crystalline portion of the total crystalline compound and may be determined by a conventional method known in the art. For example, measurement of the degree of crystallinity may be carried out by a density method or precipitation method which calculates the crystallinity degree by previous assumption of a preset value obtained by addition and/or reduction of appropriate values to/from each density of the crystalline portion and the amorphous portion, a method involving measurement of the heat of fusion, an X-ray method in which the crystallinity degree is calculated by separation of the crystalline diffraction fraction and the noncrystalline diffraction fraction from X-ray diffraction intensity distribution upon X-ray diffraction analysis, or an infrared method which calculates the crystallinity degree from a peak of the width between crystalline bands of the infrared absorption spectrum.

In the oral pharmaceutical composition according to the present invention, the crystallinity degree of the active ingredient is preferably 50% or less. More preferably, the active ingredient may have an amorphous structure from which the intrinsic crystallinity of the material was completely lost. The amorphous compound exhibits a relatively high solubility, as compared to the crystalline compound, and can significantly improve a dissolution rate and in vivo absorption rate of the drug.

In one preferred embodiment of the present invention, the amorphous structure may be formed during preparation of the active ingredient into microparticles or fine particles (micronization of the active ingredient). The microparticles may be prepared, for example by spray drying of active ingredients, melting methods involving formation of melts of active ingredients with polymers, co-precipitation involving formation of co-precipitates of active ingredients with polymers after dissolution of active ingredients in solvents, inclusion body formation, solvent volatilization, and the like. Preferred is spray drying. Even when the active ingredient is not of an amorphous structure, that is, has a crystalline structure or semi-crystalline structure, micronization of the active ingredient into fine particles via mechanical milling contributes to improvement of solubility, due to a large specific surface area of the particles, consequently resulting in improved dissolution rate and bioabsorption rate of the active drug.

The spray drying is a method of making fine particles by dissolving the active ingredient in a certain solvent and the spray-drying the resulting solution. During the spray-drying process, a high percent of the crystallinity of the naphthoquinone compound is lost to thereby result in an amorphous state, and therefore the spray-dried product in the form of a fine powder is obtained.

The mechanical milling is a method of grinding the active ingredient into fine particles by applying strong physical force to active ingredient particles. The mechanical milling may be carried out by using a variety of milling processes such as jet milling, ball milling, vibration milling, hammer milling, and the like. Particularly preferred is jet milling which can be carried out using an air pressure, at a temperature of less than 40° C.

Meanwhile, irrespective of the crystalline structure, a decreasing particle diameter of the particulate active ingredient leads to an increasing specific surface area, thereby increasing the dissolution rate and solubility. However, an excessively small particle diameter makes it difficult to prepare fine particles having such a size and also brings about agglomeration or aggregation of particles which may result in deterioration of the solubility. Therefore, in one preferred embodiment, the particle diameter of the active ingredient may be in a range of 5 nm to 500 μm. In this range, the particle agglomeration or aggregation can be maximally inhibited, and the dissolution rate and solubility can be maximized due to a high specific surface area of the particles.

Preferably, a surfactant may be additionally added to prevent the particle agglomeration or aggregation which may occur during formation of the fine particles, and/or an antistatic agent may be additionally added to prevent the occurrence of static electricity.

If necessary, a moisture-absorbent material may be further added during the milling process. The compound of Formula 1 or Formula 2 has a tendency to be crystallized by water, so incorporation of the moisture-absorbent material inhibits recrystallization of the naphthoquinone-based compound over time and enables maintenance of increased solubility of compound particles due to micronization. Further, the moisture-absorbent material serves to suppress coagulation and aggregation of the pharmaceutical composition while not adversely affecting therapeutic effects of the active ingredient.

Examples of the surfactant may include, but are not limited to, anion surfactants such as docusate sodium and sodium lauryl sulfate; cationic surfactants such as benzalkonium chloride, benzethonium chloride and cetrimide; nonionic surfactants such as glyceryl monooleate, polyoxyethylene sorbitan fatty acid ester, and sorbitan ester; amphiphilic polymers such as polyethylene-polypropylene polymer and polyoxyethylene-polyoxypropylene polymer (Poloxamer), and Gelucire™ series (Gattefosse Corporation, USA); propylene glycol monocaprylate, oleoyl macrogol-6-glyceride, linoleoyl macrogol-6-glyceride, caprylocaproyl macrogol-8-glyceride, propylene glycol monolaurate, and polyglyceryl-6-dioleate. These materials may be used alone or in any combination thereof.

Examples of the moisture-absorbent material may include, but are not limited to, colloidal silica, light anhydrous silicic acid, heavy anhydrous silicic acid, sodium chloride, calcium silicate, potassium aluminosilicate, calcium aluminosilicate, and the like. These materials may be used alone or in any combination thereof.

Some of the above-mentioned moisture absorbents may also be used as the antistatic agent.

The surfactant, antistatic agent, and moisture absorbent are added in a certain amount that is capable of achieving the above-mentioned effects, and such an amount may be appropriately adjusted depending upon micronization conditions. Preferably, the additives may be used in a range of 0.05 to 20% by weight, based on the total weight of the active ingredient

In one preferred embodiment, during formulation of the pharmaceutical composition according to the present invention into preparations for oral administration, water-soluble polymers, solubilizers and disintegration-promoting agents may be further added. Preferably, formulation of the composition into a desired dosage form may be made by mixing the additives and the particulate active ingredient in a solvent and spray-drying the mixture.

The water-soluble polymer is of help to prevent aggregation of the particulate active ingredients, by rendering surroundings of naphthoquinone-based compound molecules or particles hydrophilic to consequently enhance water solubility, and preferably to maintain the amorphous state of the active ingredient compound of Formula 1 or Formula 2.

Preferably, the water-soluble polymer is a pH-independent polymer, and can bring about crystallinity loss and enhanced hydrophilicity of the active ingredient, even under the between- and within-individual variation of the gastrointestinal pH.

Preferred examples of the water-soluble polymers may include at least one selected from the group consisting of cellulose derivatives such as methyl cellulose, hydroxymethyl cellulose, hydroxyethyl cellulose, ethyl cellulose, hydroxyethylmethyl cellulose, carboxymethyl cellulose, hydroxypropylmethyl cellulose, hydroxypropylmethyl cellulose phthalate, sodium carboxymethyl cellulose, and carboxymethylethyl cellulose; polyvinyl alcohols; polyvinyl acetate, polyvinyl acetate phthalate, polyvinylpyrrolidone (PVP), and polymers containing the same; polyalkene oxide or polyalkene glycol, and polymers containing the same. Preferred is hydroxypropylmethyl cellulose.

In the pharmaceutical composition of the present invention, an excessive content of the water-soluble polymer which is higher than a given level provides no further increased solubility, but disadvantageously brings about various problems such as overall increases in the hardness of the formulation, and non-penetration of an eluent into the formulation, by formation of films around the formulation due to excessive swelling of water-soluble polymers upon exposure to the eluent. Accordingly, the solubilizer is preferably added to maximize the solubility of the formulation by modifying physical properties of the compound of Formula 1 or Formula 2.

In this respect, the solubilizer serves to enhance solubilization and wettability of the sparingly-soluble compound of Formula 1 or Formula 2, and can significantly reduce the bioavailability variation of the naphthoquinone-based compound originating from diets and the time difference of drug administration after dietary uptake. The solubilizer may be selected from conventionally widely used surfactants or amphiphiles, and specific examples of the solubilizer may refer to the surfactants as defined above.

The disintegration-promoting agent serves to improve the drug release rate, and enables rapid release of the drug at the target site to thereby increase bioavailability of the drug.

Preferred examples of the disintegration-promoting agent may include, but are not limited to, at least one selected from the group consisting of Croscarmellose sodium, Crospovidone, calcium carboxymethylcellulose, starch glycolate sodium and lower substituted hydroxypropyl cellulose. Preferred is Croscarmellose sodium.

Upon taking into consideration various factors as described above, it is preferred to add 10 to 1000 parts by weight of the water-soluble polymer, 1 to 30 parts by weight of the disintegration-promoting agent and 0.1 to 20 parts by weight of the solubilizer, based on 100 parts by weight of the active ingredient.

In addition to the above-mentioned ingredients, other materials known in the art in connection with formulation may be optionally added, if necessary.

The solvent for spray drying is a material exhibiting a high solubility without modification of physical properties thereof and easy volatility during the spray drying process. Preferred examples of such a solvent may include, but are not limited to, dichloromethane, chloroform, methanol, and ethanol. These materials may be used alone or in any combination thereof. Preferably, a content of solids in the spray solution is in a range of 5 to 50% by weight, based on the total weight of the spray solution.

The above-mentioned intestine-targeted formulation process may be preferably carried out for formulation particles prepared as above.

In one preferred embodiment, the oral pharmaceutical composition according to the present invention may be formulated by a process comprising the following steps:

(a) adding the compound of Formula 1 or Formula 2 alone or in combination with a surfactant and a moisture-absorbent material, and grinding the compound of Formula 1 with a jet mill to prepare active ingredient microparticles;

(b) dissolving the active ingredient microparticles in conjunction with a water-soluble polymer, a solubilizer and a disintegration-promoting agent in a solvent and spray-drying the resulting solution to prepare formulation particles; and

(c) dissolving the formulation particles in conjunction with a pH-sensitive polymer and a plasticizer in a solvent and spray-drying the resulting solution to carry out intestine-targeted coating on the formulation particles.

The surfactant, moisture-absorbent material, water-soluble polymer, solubilizer and disintegration-promoting agent are as defined above. The plasticizer is an additive added to prevent hardening of the coating, and may include, for example polymers such as polyethylene glycol.

Alternatively, formulation of the active ingredient may be carried out by sequential or concurrent spraying of vehicles of step (b) and intestine-targeted coating materials of step (c) onto jet-milled active ingredient particles of step (a) as a seed.

Meanwhile, for injection, the agents of the present invention may be formulated in aqueous solutions, preferably in physiologically compatible buffers such as Hanks's solution, Ringer's solution, or physiological saline. For transmucosal administration, penetrants appropriate to the bather to be permeated are used in the formulation. Such penetrants are generally known in the art.

The compounds may be formulated for parenteral administration by injection, e.g., by bolus injection or continuous infusion. Formulations for injection may be presented in unit dosage forms, e.g., in ampoules or in multi dose containers, with an added preservative. The compositions may take such forms as suspensions, solutions or emulsions in oily or aqueous vehicles, and may contain formulatory agents such as suspending, stabilizing or dispersing agents.

Alternatively, the active ingredient may be in powder form for constitution with a suitable vehicle, e.g., sterile pyrogen-free water, before use.

Pharmaceutical compositions suitable for use in the present invention include compositions in which the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

When the pharmaceutical composition of the present invention is formulated into a unit dosage form, the compound of Formula 1 or Formula 2 as the active ingredient is preferably contained in a unit dose of about 0.1 to 1,000 mg. The amount of the compound of Formula 1 or Formula 2 administered will be determined by the attending physician, depending upon body weight and age of patients being treated, characteristic nature and the severity of diseases.

In accordance with another aspect of the present invention, there is provided use of the compound of Formula 1 or Formula 2 in the preparation of a drug for the treatment or prevention of erectile dysfunction. The term “treatment” of the disease syndromes refers to stopping or delaying of the disease progress, when the drug is used in the subject exhibiting symptoms of disease onset. The term “prevention” refers to stopping or delaying of symptoms of disease onset, when the drug is used in the subject exhibiting no symptoms of disease onset but having high risk of disease onset

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a view showing factors determining activity of AMP-activated protein kinase (AMPK) and results obtained from the AMPK activity;

FIG. 2 is a graph comparing relaxation effects of penile corpus cavernosum smooth muscles between the groups with administration of pharmaceutically active ingredients of conventional erectile dysfunction drugs, compounds acting as AMPK activators and the compound 1;

FIG. 3 is a view showing influence of the compound 1 on phosphorylation of eNOS;

FIG. 4 is a graph comparing relaxation inhibition effects between the L-NAME-treatment group and the methylene blue-treatment group after relaxation of penile corpus cavernosum smooth muscles induced by the compound 1;

FIGS. 5 and 6 are graphs comparing inhibitory effects on relaxation of penile corpus cavernosum smooth muscles between the group with administration of CHAPS (10⁻⁴M), and the group with administration of CHAPS and zinc-protoporphyrin-IX (ZnPP), after relaxation of penile corpus cavernosum smooth muscles induced by the compound 1; and

FIG. 7 is a graph showing an increase in internal pressure of penile corpus cavernosum smooth muscles of diabetes-induced rats, to which and the compound 1 are orally administered, between Groups I, II, III and IV.

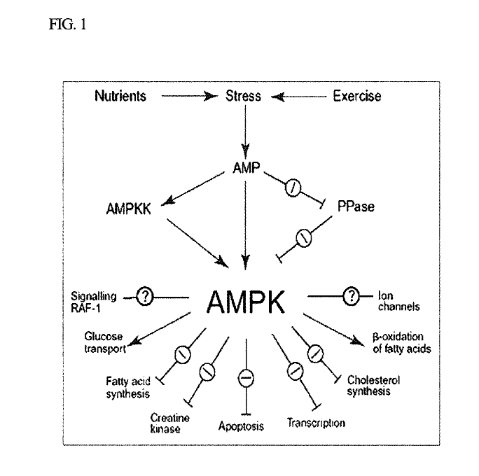

### DETAILED DESCRIPTION OF PREFERRED EMBODIMENTS

Now, the present invention will be described in more detail with reference to the following Examples and Experimental Examples. These examples are provided only for illustrating the present invention and should not be construed as limiting the scope and spirit of the present invention.

### Example 1

### Synthesis of β-lapachone (Compound 1)

17.4 g (0.10M) of 2-hydroxy-1,4-naphthoquinone was dissolved in 120 ml of DMSO, and 0.88 g (0.11M) of LiH was gradually added thereto. Here, this should be done with care because hydrogen evolves. The reaction solution was stirred, and after confirming no further production of hydrogen, was additionally stirred for another 30 min. Then, 15.9 g (0.10M) of prenyl bromide (1-bromo-3-methyl-2-butene) and 3.35 g (0.025M) of LiI were gradually added thereto. The reaction solution was heated to 45° C. and then stirred vigorously for 12 hours at that temperature. The reaction solution was cooled below 10° C., and 76 g of ice was first added and 250 ml of water was then added. Thereafter, 25 ml of concentrated HCl was gradually added to maintain the resulting solution at an acidic pH>1. 200 ml of EtOAc was added to the reaction mixture which was then stirred vigorously, thereby producing white solids that were not dissolved in EtOAc. These solids were filtered and an EtOAc layer was separated. The aqueous layer was extracted once again with 100 ml of EtOAc and was combined with the previously extracted organic layer. The organic layer was washed with 150 ml of 5% NaHCO₃, and was concentrated. The resulting concentrates were dissolved in 200 ml of CH₂Cl₂, and were vigorously shaken to separate two layers with addition of 70 ml of an aqueous 2N NaOH solution. A CH₂Cl₂ layer was further separated twice with treatment of an aqueous 2N NaOH solution (70 ml×2). The thus-separated aqueous solutions were combined together and adjusted to an acidic pH>2, thereby forming solids. The resulting solids were filtered and separated to give Lapachol. The thus-obtained Lapachol was recrystallized from 75% EtOH. The resulting Lapachol was mixed with 80 ml of sulfuric acid, and the mixture was vigorously stirred at room temperature for 10 min and 200 g of ice was added thereto to complete the reaction. 60 ml of CH₂Cl₂ was added to the reaction materials which were then shaken vigorously. Thereafter, a CH₂Cl₂ layer was separated and washed with 5% NaHCO₃. An aqueous layer was extracted once again using 30 ml of CH₂Cl₂, washed with 5% NaHCO₃ and combined with the previously extracted organic layer. The organic layer was dried over MgSO₄ and concentrated to give impure β-Lapachone. The thus-obtained β-Lapachone was recrystallized from isopropanol, thereby obtaining 8.37 g of pure β-Lapachone.

¹H-NMR (CDCl₃, δ): 8.05 (1H, dd, J=1, 8 Hz), 7.82 (1H, dd, J=1, 8 Hz), 7.64 (1H, dt, J=1, 8 Hz), 7.50 (1H, dt, J=1, 8 Hz), 2.57 (2H, t, J=6.5 Hz), 1.86 (2H, t, J=6.5 Hz) 1.47 (6H, s)

### Example 2

### Synthesis of Dunnione (Compound 2)

In the process of obtaining Lapachol in Example 1, solids separated without being dissolved in EtOAc are 2-prenyloxy-1,4-naphthoquinone, an O-alkylation product, unlike Lapachol which is a C-alylation product. The separated 2-prenyloxy-1,4-naphthoquinone was first recrystallized once again from EtOAc. 3.65 g (0.015M) of the thus-purified solids was dissolved in toluene and toluene was refluxed for 5 hours to induce Claisen Rearrangement. Toluene was concentrated by distillation under reduced pressure and was then mixed with 15 ml of sulfuric acid, without further purification. The resulting mixture was stirred vigorously at room temperature for 10 min and 100 g of ice was added thereto to complete the reaction. 50 ml of CH₂Cl₂ was added to the reaction materials which were shaken vigorously. Thereafter, a CH₂Cl₂ layer was separated and washed with 5% NaHCO₃. An aqueous layer was extracted once again using 20 ml of CH₂Cl₂, washed with 5% NaHCO₃ and combined with the previously extracted organic layer. The organic layer was dried over MgSO₄, concentrated and purified by chromatography on silica gel to give 2.32 g of pure Dunnione.

¹H-NMR (CDCl₃, δ): 8.05 (1H, d, J=8 Hz), 7.64 (2H, d, J=8 Hz), 7.56 (1H, m), 4.67 (1H, q, J=7 Hz), 1.47 (3H, d, J=7 Hz), 1.45 (3H, s) 1.27 (3H, s)

### Example 3

### Synthesis of α-Dunnione (Compound 3)

4.8 g (0.020M) of 2-prenyloxy-1,4-naphthoquinone purified in Example 2 was dissolved in xylene, and xylene was refluxed for 15 hours, thereby inducing Claisen Rearrangement under significantly higher temperature conditions and prolonged reaction conditions as compared to Example 2. According to this reaction process, α-Dunnione that had progressed to cyclization was obtained together with a Lapachol derivative which had undergone Claisen Rearrangement and in which one of two methyl groups has shifted. Xylene was concentrated by distillation under reduced pressure and purified by chromatography on silica gel to give 1.65 g of pure α-Dunnione.

¹H-NMR (CDCl₃, δ): 8.06 (1H, d, J=8 Hz), 7.64 (2H, m), 7.57 (1H, m), 3.21 (1H, q, J=7 Hz), 1.53 (3H, s), 1.51 (3H, s) 1.28 (3H, d, J=7 Hz)

### Example 4

### Synthesis of Compound 4

17.4 g (0.10M) of 2-hydroxy-1,4-naphthoquinone was dissolved in 120 ml of DMSO, and 0.88 g (0.11M) of LiH was gradually added thereto. Here, this should be done with care because hydrogen evolves. The reaction solution was stiffed, and after confirming no further production of hydrogen, was additionally stirred for another 30 min. Then, 14.8 g (0.11M) of methallyl bromide (1-bromo-2-methylpropene) and 3.35 g (0.025M) of LiI were gradually added thereto. The reaction solution was heated to 45° C. and then stirred vigorously for 12 hours at that temperature. The reaction solution was cooled below 10° C., and 80 g of ice was first added and 250 ml of water was then added. Thereafter, 25 ml of concentrated HCl was gradually added to maintain the resulting solution at an acidic pH>1.200 ml of CH₂Cl₂ was added to the reaction mixture which was then shaken vigorously to separate two layers. The aqueous layer was extracted once again with addition of 70 ml of CH₂Cl₂ and was combined with the previously extracted organic layer. Two materials were confirmed to be formed newly by TLC and were subsequently used without any particular separation process. The organic layer was concentrated by distillation under reduced pressure, dissolved again in xylene and then refluxed for 8 hours. In this process, two materials on TLC were combined into one, thereby obtaining a relatively pure Lapachol derivative. The thus-obtained Lapachol derivative was mixed with 80 ml of sulfuric acid and stirred vigorously at room temperature for 10 min, and 200 g of ice was added thereto to complete the reaction. 80 ml of CH₂Cl₂ was added to the reaction materials which were then shaken vigorously. Thereafter, a CH₂Cl₂ layer was separated and washed with 5% NaHCO₃. An aqueous layer was extracted once again using 50 ml of CH₂Cl₂, washed with 5% NaHCO₃ and combined with the previously extracted organic layer. The organic layer was dried over MgSO₄ and concentrated to give impure β-Lapachone derivative (Compound 4). The thus-obtained β-Lapachone derivative was recrystallized from isopropanol, thereby obtaining 12.21 g of pure Compound 4.

¹H-NMR (CDCl₃, δ): 8.08 (1H, d, J=8 Hz), 7.64 (2H, m), 7.57 (1H, m), 2.95 (2H, s), 1.61 (6H, s)

### Example 5

### Synthesis of Compound 5

Compound 5 was obtained in the same manner as in Example 4, except that allyl bromide was used instead of methallyl bromide.

¹H-NMR (CDCl₃, δ): 8.07 (1H, d, J=7 Hz), 7.65 (2H, m), 7.58 (1H, m), 5.27 (1H, m), 3.29 (1H, dd, J=10, 15 Hz), 2.75 (1H, dd, J=7, 15 Hz), 1.59 (3H, d, J=6 Hz)

### Example 6

### Synthesis of Compound 6

5.08 g (40 mM) of 3-chloropropionyl chloride was dissolved in 20 ml of ether and cooled to −78° C. 1.95 g (25 mM) of sodium peroxide (Na₂O₂) was gradually added to the resulting solution while being vigorously stirred at that temperature, followed by further vigorous stirring for 30 min. The reaction solution was heated to 0° C. and 7 g of ice was added thereto, followed by additional stirring for another 10 min. An organic layer was separated, washed once again with 10 ml of cold water at 0° C., then with an aqueous NaHCO₃ solution at 0° C. The organic layer was separated, dried over MgSO₄, concentrated by distillation under reduced pressure below 0° C., thereby preparing 3-chloropropionic peracid.

1.74 g (10 mM) of 2-hydroxy-1,4-naphthoquinone was dissolved in 20 ml of acetic acid, and the previously prepared 3-chloropropionic peracid was gradually added thereto at room temperature. The reaction mixture was refluxed with stirring for 2 hours, and then distilled under reduced pressure to remove acetic acid. The resulting concentrates were dissolved in 20 ml of CH₂Cl₂, and washed with 20 ml of 5% NaHCO₃. An aqueous layer was extracted once again using 20 ml of CH₂Cl₂ and combined with the previously extracted organic layer. The organic layer was dried over MgSO₄ and concentrated to give Compound 6 in admixture with 2-(2-chloroethyl)-3-hydroxy-1,4-naphthoquinone. The resulting mixture was purified by chromatography on silica gel to give 0.172 g of a pure Lapachone derivative (Compound 6).

¹H-NMR (CDCl₃, δ): 8.07 (1H d, J=7.6 Hz), 7.56-7.68 (3H, m), 4.89 (2H, t, J=9.2 Hz), 3.17 (2H, t, J=9.2 Hz)

### Example 7

### Synthesis of Compound 7

17.4 g (0.10M) of 2-hydroxy-1,4-naphthoquinone was dissolved in 120 ml of DMSO, and 0.88 g (0.11M) of LiH was gradually added thereto. Here, this should be done with care because hydrogen evolves. The reaction solution was stirred, and after confirming no further production of hydrogen, was additionally stirred for another 30 min. Then, 19.7 g (0.10M) of cinnamyl bromide (3-phenylephrine nylallyl bromide) and 3.35 g (0.025M) of LiI were gradually added thereto. The reaction solution was heated to 45° C. and then stirred vigorously for 12 hours at that temperature. The reaction solution was cooled below 10° C., and 80 g of ice was first added and 250 ml of water was then added. Thereafter, 25 ml of concentrated HCl was gradually added to maintain the resulting solution at an acidic pH>1. 200 ml of CH₂Cl₂ was added to dissolve the reaction mixture which was then shaken vigorously to separate two layers. The aqueous layer was discarded, and a CH₂Cl₂ layer was treated with an aqueous 2N NaOH solution (100 ml×2) to separate the aqueous layer twice. At this time, the remaining CH₂Cl₂ layer after extraction with an aqueous 2N NaOH solution was used again in Example 8. The thus-separated aqueous solutions were combined and adjusted to an acidic pH>2 using concentrated HCl, thereby forming solids. The resulting solids were filtered and separated to give a Lapachol derivative. The thus-obtained Lapachol derivative was recrystallized from 75% EtOH. The resulting Lapachol derivative was mixed with 50 ml of sulfuric acid, and the mixture was vigorously stirred at room temperature for 10 min and 150 g of ice was added thereto to complete the reaction. 60 ml of CH₂Cl₂ was added to the reaction materials which were then shaken vigorously. Thereafter, a CH₂Cl₂ layer was separated and washed with 5% NaHCO₃. An aqueous layer was extracted once again using 30 ml of CH₂Cl₂, washed with 5% NaHCO₃ and combined with the previously extracted organic layer. The organic layer was concentrated and purified by chromatography on silica gel to give 2.31 g of pure Compound 7.

¹H-NMR (CDCl₃, δ): 8.09 (1H, dd, J=1.2, 7.6 Hz), 7.83 (1H, d, J=7.6 Hz), 7.64 (1H, dt, J=1.2, 7.6 Hz), 7.52 (1H, dt, J=1.2, 7.6 Hz), 7.41 (5H, m), 5.27 (1H, dd, J=2.5, 6.0 Hz), 2.77 (1H, m) 2.61 (1H, m), 2.34 (1H, m), 2.08 (1H, m), 0.87 (1H, m)

### Example 8

### Synthesis of Compound 8

The remaining CH₂Cl₂ layer, after extraction with an aqueous 2N NaOH solution in Example 7, was concentrated by distillation under reduced pressure. The resulting concentrates were dissolved in 30 ml of xylene, followed by reflux for 10 hours to induce Claisen Rearrangement Xylene was concentrated by distillation under reduced pressure and was then mixed with 15 ml of sulfuric acid, without further purification. The resulting mixture was stirred vigorously at room temperature for 10 min and 100 g of ice was added thereto to complete the reaction. 50 ml of CH₂Cl₂ was added to the reaction materials which were shaken vigorously. Thereafter, a CH₂Cl₂ layer was separated and washed with 5% NaHCO₃. An aqueous layer was extracted once again using 20 ml of CH₂Cl₂, washed with 5% NaHCO₃ and combined with the previously extracted organic layer. The organic layer was dried over MgSO₄, concentrated and purified by chromatography on silica gel to give 1.26 g of pure Compound 8.

¹H-NMR (CDCl₃, δ): 8.12 (1H, dd, J=0.8, 8.0 Hz), 7.74 (1H, dd, J=12, 7.6 Hz), 7.70 (1H, dt, J=1.2, 7.6 Hz), 7.62 (1H, dt, J=1.6, 7.6 Hz), 7.27 (3H, m), 7.10 (2H, td, J=1.2, 6.4 Hz), 5.38 (1H, qd, J=6.4, 9.2 Hz), 4.61 (1H, d, J=9.2 Hz), 1.17 (3H, d, J=6.4 Hz)

### Example 9

### Synthesis of Compound 9

3.4 g (22 mM) of 1,8-diazabicyclo[5.4.0]undec-7-ene and 1.26 g (15 mM) of 2-methyl-3-butyn-2-ol were dissolved in 10 ml of acetonitrile and the resulting solution was cooled to 0° C. 3.2 g (15 mM) of trifluoroacetic anhydride was gradually added with stirring to the reaction solution which was then continued to be stirred at 0° C. 1.74 g (10 mM) of 2-hydroxy-1,4-naphthoquinone and 135 mg (1.0 mM) of cupric chloride (CuCl₂) were dissolved in 10 ml of acetonitrile in another flask, and were stirred. The previously purified solution was gradually added to the reaction solution which was then refluxed for 20 hours. The reaction solution was concentrated by distillation under reduced pressure and was then purified by chromatography on silica gel to give 0.22 g of pure Compound 9.

¹H-NMR (CDCl₃, δ): 8.11 (1H, dd, J=1.2, 7.6 Hz), 7.73 (1H, dd, J=1.2, 7.6 Hz), 7.69 (1H, dt, J=1.2, 7.6 Hz), 7.60 (1H, dt, J=1.6, 7.6 Hz), 4.95 (1H, d, J=3.2 Hz), 4.52 (1H, d, J=3.2 Hz), 1.56 (6H, s)

### Example 10

### Synthesis of Compound 10

0.12 g of Compound 9 was dissolved in 5 ml of MeOH, 10 mg of 5% Pd/C was added thereto, followed by vigorous stirring at room temperature for 3 hours. The reaction solution was filtered through silica gel to remove 5% Pd/C and was concentrated by distillation under reduced pressure to give Compound 10.

¹H-NMR (CDCl₃, δ): 8.05 (1H, td, J=1.2, 7.6 Hz), 7.64 (2H, m), 7.54 (1H, m), 3.48 (3H, s), 1.64 (3H, s), 1.42 (3H, s), 1.29 (3H, s)

### Example 11

### Synthesis of Compound 11

1.21 g (50 mM) of β-Lapachone (Compound 1) and 1.14 g (50 mM) of DDQ (2,3-dichloro-5,6-dicyano-1,4-benzoqinone) were dissolved in 50 ml of carbon tetrachloride and refluxed for 72 hours. The reaction solution was concentrated by distillation under reduced pressure and was then purified by chromatography on silica gel to give 1.18 g of pure Compound 11.

¹H-NMR (CDCl₃, δ): 8.08 (1H, dd, J=1.2, 7.6 Hz), 7.85 (1H, dd, 7.6 Hz), 7.68 (1H, dt, J=1.2, 7.6 Hz), 7.55 (1H, dt, 7.6 Hz), 6.63 (1H, d, J=10.0 Hz), 5.56 (1H, d, J=10.0 Hz), 1.57 (6H, s)

### Example 12

### Synthesis of Compound 12

1.74 g (10 mM) of 2-hydroxy-1,4-naphthoquinone, 3.4 g (50 mM) of 2-methyl-1,3-butadiene (Isoprene), 3.0 g (100 mM) of paraformaldehyde and 20 ml of 1,4-dioxane were placed into a pressure vessel, and were heated with stirring at 100° C. for 48 hours. The reaction vessel was cooled to room temperature, and contents therein were filtered. The filtrate was concentrated by distillation under reduced pressure and was then purified by chromatography on silica gel to give 238 mg of Compound 12, as a 2-vinyl derivative off β-Lapachone.

¹H-NMR (CDCl₃, δ): 8.07 (1H, dd, J=1.2, 7.6 Hz), 7.88 (1H, dd, 7.6 Hz), 7.66 (1H, dt, J=1.2, 7.6 Hz), 7.52 (1H, dt, J=0.8, 7.6 Hz), 5.87 (1H, dd, J=10.8, 17.2 Hz), 5.18 (1H d, J=10.8 Hz), 5.17 (1H, 17.2 Hz), 2.62 (1H, m), 2.38 (1H, m), 2.17 (3H, s), 2.00 (1H, m), 1.84 (1H, m)

### Example 13

### Synthesis of Compound 13

1.74 g (10 mM) of 2-hydroxy-1,4-naphthoquinone, 4.8 g (50 mM) of 2,4-dimethyl-1,3-pentadiene and 3.0 g (100 mM) of paraformaldehyde were dissolved in 20 ml of 1,4-dioxane, and the resulting mixture was refluxed with vigorous stirring for 10 hours. The reaction vessel was cooled to room temperature, and contents therein were filtered to remove paraformaldehyde from solids. The filtrate was concentrated by distillation under reduced pressure and was then purified by chromatography on silica gel to give 428 mg of Compound 13, as a β-Lapachone derivative.

¹H-NMR (CDCl₃, δ): 8.06 (1H, dd, J=1.2, 7.6 Hz), 7.83 (1H, dd, J=0.8, 7.6 Hz), 7.65 (1H, dt, J=1.2, 7.6 Hz), 7.50 (1H, dt, 7.6 Hz), 5.22 (1H, bs), 2.61 (1H, m), 2.48 (1H, m), 2.04 (1H, m), 1.80 (3H, d, J=1.0 Hz), 1.75 (1H, m), 1.72 (1H, d, J=1.0 Hz), 1.64 (3H, s)

### Example 14

### Synthesis of Compound 14

5.3 g (30 mM) of 2-hydroxy-1,4-naphthoquinone, 20.4 g (150 mM) of 2,6-dimethyl-2,4,6-octatriene and 9.0 g (300 mM) of paraformaldehyde were dissolved in 50 ml of 1,4-dioxane, and the resulting mixture was refluxed with vigorous stirring for 10 hours. The reaction vessel was cooled to room temperature, and contents therein were filtered to remove paraformaldehyde from solids. The filtrate was concentrated by distillation under reduced pressure and was then purified by chromatography on silica gel to give 1.18 g of Compound 14, as a β-Lapachone derivative.

¹H-NMR (CDCl₃, δ): 8.07 (1H, dd, J=1.2, 7.6 Hz), 7.87 (1H, dd, J=0.8, 7.6 Hz), 7.66 (1H, dt, J=1.2, 7.6 Hz), 7.51 (1H, dt, J=0.8, 7.6 Hz), 6.37 (1H, dd, J=11.2, 15.2 Hz), 5.80 (1H, broad d, J=11.2 Hz), 5.59 (1H, d, J=15.2 Hz), 2.67 (1H, dd, J=4.8, 17.2 Hz), 2.10 (1H, dd, J=6.0, 17.2 Hz), 1.97 (1H, m), 1.75 (3H, bs), 1.64 (311, bs), 1.63 (3H, s), 1.08 (3H, d, J=6.8 Hz)

### Example 15

### Synthesis of Compound 15

5.3 g (30 mM) of 2-hydroxy-1,4-naphthoquinone, 20.4 g (50 mM) of terpinen and 9.0 g (300 mM) of paraformaldehyde were dissolved in 50 ml of 1,4-dioxane, and the resulting mixture was refluxed with vigorous stirring for 10 hours. The reaction vessel was cooled to room temperature, and contents therein were filtered to remove paraformaldehyde from solids. The filtrate was concentrated by distillation under reduced pressure and was then purified by chromatography on silica gel to give 1.12 g of Compound 15, as a tetracyclic o-quinone derivative.

¹H-NMR (CDCl₃, δ): 8.06 (1H, d, J=7.6 Hz), 7.85 (1H, d, J=7.6 Hz), 7.65 (1H, t, J=7.6 Hz), 7.51 (1H, t, J=7.6 Hz), 5.48 (1H, broad s), 4.60 (1H, broad s), 2.45 (1H, d, J=16.8 Hz), 2.21 (1H, m), 2.20 (1H, d, J=16.8 Hz), 2.09 (1H, m), 1.77 (1H, m), 1.57 (1H, m), 1.07 (3H, s), 1.03 (3H, d, J=0.8 Hz), 1.01 (3H, d, J=0.8 Hz), 0.96 (1H, m)

### Example 16

### Synthesis of Compounds 16 and 17

17.4 g (0.10M) of 2-hydroxy-1,4-naphthoquinone was dissolved in 120 ml of DMSO, and 0.88 g (0.11M) of LiH was gradually added thereto. Here, this should be done with care because hydrogen evolves. The reaction solution was stirred, and after confirming no further production of hydrogen, was additionally stirred for another 30 min. Then, 16.3 g (0.12M) of crotyl bromide and 3.35 g (0.025M) of LiI were gradually added thereto. The reaction solution was heated to 45° C. and then vigorously stirred for 12 hours at that temperature. The reaction solution was cooled below 10° C., and 80 g of ice was first added and 250 ml of water was then added. Thereafter, 25 ml of concentrated HCl was gradually added to maintain the resulting solution at an acidic pH>1.200 ml of CH₂Cl₂ was added to dissolve the reaction mixture which was then shaken vigorously to separate two layers. The aqueous layer was discarded, and a CH₂Cl₂ layer was treated with an aqueous 2N NaOH solution (100 ml×2) to separate the aqueous layer twice. At this time, the remaining CH₂Cl₂ layer after extraction with an aqueous 2N NaOH solution was used in Example 17. The thus-separated aqueous solutions were combined and adjusted to an acidic pH>2 using concentrated HCl, thereby forming solids. The resulting solids were filtered and separated to give a Lapachol derivative. The thus-obtained Lapachol derivative was recrystallized from 75% EtOH. The resulting Lapachol derivative was mixed with 50 ml of sulfuric acid, and the mixture was vigorously stirred at room temperature for 10 min, followed by addition of 150 g of ice to complete the reaction. 60 ml of CH₂Cl₂ was added to the reaction materials which were then shaken vigorously. Thereafter, a CH₂Cl₂ layer was separated and washed with 5% NaHCO₃. An aqueous layer was extracted once again using 30 ml of CH₂Cl₂, washed with 5% NaHCO₃ and combined with the previously extracted organic layer. The organic layer was concentrated and purified by chromatography on silica gel to give 1.78 g and 0.43 g of pure Compounds 16 and 17, respectively.

¹H-NMR (CDCl₃, δ) of Compound 16: δ8.07 (1H, dd, J=0.8, 6.8 Hz), 7.64 (2H, broad d, J=3.6 Hz), 7.57 (1H, m), 5.17 (1H, qd, J=6.0, 8.8 Hz), 3.53 (1H, qd, J=6.8, 8.8 Hz), 1.54 (3H, d, 6.8 Hz), 1.23 (3H, d, 6.8 Hz)

¹H-NMR (CDCl₃, δ) of Compound 17: δ8.06 (1H, d, J=0.8, 7.2 Hz), 7.65 (2H, broad d, J=3.6 Hz), 7.57 (1H, m), 4.71 (1H, quintet, J=6.4 Hz), 3.16 (1H, quintet, J=6.4 Hz), 1.54 (3H, d, 6.4 Hz), 1.38 (3H, d, 6.4 Hz)

### Example 17

### Synthesis of Compounds 18 and 19

The remaining CH₂Cl₂ layer, after extraction with an aqueous 2N NaOH solution in Example 16, was concentrated by distillation under reduced pressure. The resulting concentrates were dissolved in 30 ml of xylene, followed by reflux for 10 hours to induce Claisen Rearrangement. Xylene was concentrated by distillation under reduced pressure and was then mixed with 15 ml of sulfuric acid, without further purification. The resulting mixture was stirred vigorously at room temperature for 10 min and 100 g of ice was added thereto to complete the reaction. 50 ml of CH₂Cl₂ was added to the reaction materials which were shaken vigorously. Thereafter, a CH₂Cl₂ layer was separated and washed with 5% NaHCO₃. An aqueous layer was extracted once again using 20 ml of CH₂Cl₂, washed with 5% NaHCO₃ and combined with the previously extracted organic layer. The organic layer was dried over MgSO₄, concentrated and purified by chromatography on silica gel to give 0.62 g and 0.43 g of pure Compounds 18 and 19, respectively.

¹H-NMR (CDCl₃, δ) of Compound 18: 8.06 (1H, dd, J=0.8, 7.2 Hz), 7.81 (1H, dd, J=0.8, 7.6 Hz), 7.65 (1H, dt, J=0.8, 7.6 Hz), 7.51 (1H, dt, J=0.8, 7.2 Hz), 4.40 (1H, m), 2.71 (1H, m), 2.46 (1H, m), 2.11 (1H, m), 1.71 (1H, m), 1.54 (3H, d, 6.4 Hz), 1.52 (1H, m)

¹H-NMR (CDCl₃, δ) of Compound 19: 8.08 (1H, d, J=0.8, 7.2 Hz), 7.66 (2H, broad d, J=4.0 Hz), 7.58 (1H, m), 5.08 (1H, m), 3.23 (1H, dd, J=9.6, 15.2 Hz), 2.80 (1H, dd, J=7.2, 15.2 Hz), 1.92 (1H, m), 1.82 (1H, m), 1.09 (3H, t, 7.6 Hz)

### Example 18

### Synthesis of Compound 20

17.4 g (0.10M) of 2-hydroxy-1,4-naphthoquinone was dissolved in 120 ml of DMSO, and 0.88 g (0.11M) of LiH was gradually added thereto. Here, this should be done with care because hydrogen evolves. The reaction solution was stirred, and after confirming no further production of hydrogen, was additionally stirred for another 30 min. Then, 21.8 g (0.10M) of geranyl bromide and 3.35 g (0.025M) of LiI were gradually added thereto. The reaction solution was heated to 45° C. and then vigorously stirred for 12 hours at that temperature. The reaction solution was cooled below 10° C., and 80 g of ice was first added and 250 ml of water was then added. Thereafter, 25 ml of concentrated HCl was gradually added to maintain the resulting solution at an acidic pH>1. 200 ml of CH₂Cl₂ was added to dissolve the reaction mixture which was then shaken vigorously to separate two layers. The aqueous layer was discarded, and a CH₂Cl₂ layer was treated with an aqueous 2N NaOH solution (100 ml×2) to separate the aqueous layer twice. The thus-separated aqueous solutions were combined and adjusted to an acidic pH>2 using concentrated HCl, thereby forming solids. The resulting solids were filtered and separated to give 2-geranyl-3-hydroxy-1,4-naphthoquinone. The thus-obtained product was mixed with 50 ml of sulfuric acid without further purification, and the mixture was vigorously stirred at room temperature for 10 min, followed by addition of 150 g of ice to complete the reaction. 60 ml of CH₂Cl₂ was added to the reaction materials which were then shaken vigorously. Thereafter, a CH₂Cl₂ layer was separated and washed with 5% NaHCO₃. An aqueous layer was extracted once again using 30 ml of CH₂Cl₂, washed with 5% NaHCO₃ and combined with the previously extracted organic layer. The organic layer was concentrated and purified by chromatography on silica gel to give 3.62 g of pure Compound 20.

¹H-NMR (CDCl₃, δ): 8.05 (1H, d, J=7.6 Hz), 7.77 (1H, d, J=7.6 Hz), 7.63 (1H, t, J=7.6 Hz), 7.49 (1H, t, J=7.6 Hz), 2.71 (1H, dd, J=6.0, 17.2 Hz), 2.19 (1H, dd, J=12.8, 17.2 Hz), 2.13 (1H, m), 1.73 (2H, m), 1.63 (1H, dd, J=6.0, 12.8 Hz), 1.59 (1H, m), 1.57 (1H, m), 1.52 (1H, m), 1.33 (3H, s), 1.04 (3H, s), 0.93 (3H, s)

### Example 19

### Synthesis of Compound 21

Compound 21 was obtained in the same manner as in Example 1, except that 6-chloro-2-hydroxy-1,4-naphthoquinone was used instead of 2-hydroxy-1,4-naphthoquinone.

¹H-NMR (CDCl₃, δ): 8.02 (1H, d, J=8 Hz), 7.77 (1H, d, J=2 Hz), 7.50 (1H, dd, J=2, 8 Hz), 2.60 (2H, t, J=7 Hz), 1.87 (2H, t, J=7 Hz) 1.53 (6H, s)

### Example 20

### Synthesis of Compound 22

Compound 22 was obtained in the same manner as in Example 1, except that 2-hydroxy-6-methyl-1,4-naphthoquinone was used instead of 2-hydroxy-1,4-naphthoquinone.

¹H-NMR (CDCl₃, δ): 7.98 (1H, d, J=8 Hz), 7.61 (1H, d, J=2 Hz), 7.31 (1H, dd, J=2, 8 Hz), 2.58 (2H, t, J=7 Hz), 1.84 (214, t, J=7 Hz) 1.48 (6H, s)

### Example 21

### Synthesis of Compound 23

Compound 23 was obtained in the same manner as in Example 1, except that 6,7-dimethoxy-2-hydroxy-1,4-naphthoquinone was used instead of 2-hydroxy-1,4-naphthoquinone.

¹H-NMR. (CDCl₃, δ): 7.56 (1H, s), 7.25 (1H, s), 3.98 (6H, s), 2.53 (2H, t, J=7′Hz), 1.83 (2H, t, J=7 Hz) 1.48 (6H, s)

### Example 22

### Synthesis of Compound 24

Compound 24 was obtained in the same manner as in Example 1, except that 1-bromo-3-methyl-2-pentene was used instead of 1-bromo-3-methyl-2-butene.

¹H-NMR (CDCl₃, δ): 7.30-8.15 (4H, m), 2.55 (2H, t, J=7 Hz), 1.83 (2H, t, J=7 Hz), 1.80 (2H, q, 7 Hz) 1.40 (3H, s), 1.03 (3H, t, J=7 Hz)

### Example 23

### Synthesis of Compound 25

Compound 25 was obtained in the same manner as in Example 1, except that 1-bromo-3-ethyl-2-pentene was used instead of 1-bromo-3-methyl-2-butene.

¹H-NMR (CDCl₃, δ): 7.30-8.15 (4H, m), 2.53 (2H, t, J=7 Hz), 1.83 (2H, t, J=7 Hz), 1.80 (4H, q, 7 Hz) 0.97 (6H, t, J=7 Hz)

### Example 24

### Synthesis of Compound 26

Compound 26 was obtained in the same manner as in Example 1, except that 1-bromo-3-phenylephrinenyl-2-butene was used instead of 1-bromo-3-methyl-2-butene.

¹H-NMR (CDCl₃, δ): 7.15-8.15 (9H, m), 1.90-2.75 (4H, m), 1.77 (3H, s)

### Example 25

### Synthesis of Compound 27

Compound 27 was obtained in the same manner as in Example 1, except that 2-bromo-ethylidenecyclohexane was used instead of 1-bromo-3-methyl-2-butene.

¹H-NMR (CDCl₃, δ): 7.30-8.25 (4H, m), 2.59 (2H, t, J=7 Hz), 1.35-2.15 (12H, m)

### Example 26

### Synthesis of Compound 28

Compound 28 was obtained in the same manner as in Example 1, except that 2-bromo-ethylidenecyclopentane was used instead of 1-bromo-3-methyl-2-butene.

¹H-NMR (CDCl₃, δ): 7.28-8.20 (4H, m), 2.59 (2H, t, J=7 Hz), 1.40-2.20 (10H, m)

### Example 27

### Synthesis of Compound 29

8.58 g (20 mM) of Compound 5 synthesized in Example 5 was dissolved in 1000 ml of carbon tetrachloride, followed by addition of 11.4 g (50 mM) of 2,3-dichloro-5,6-dicyano-1,4-benzoqinone, and the resulting mixture was refluxed for 96 hours. The reaction solution was concentrated by distillation under reduced pressure and the resulting red solids were then recrystallized from isopropanol, thereby obtaining 7.18 g of pure Compound 29.

¹H-NMR (CDCl₃, δ): 8.05 (1H, dd, J=1.2, 7.6 Hz), 7.66 (1H, dd, J=1.2, 7.6 Hz), 7.62 (1H, dt, J=1.2, 7.6 Hz), 7.42 (1H, dt, J=1.2, 7.6 Hz), 6.45 (1H, q, J=1.2 Hz), 2.43 (3H, d, J=1.2 Hz)

### Example 28

### Synthesis of Compound 30

Analogous to a synthesis method as taught in J. Org. Chem., 55 (1990) 4995-5008, 4,5-dihydro-3-methylbenzo[1,2-b]furan-4,5-dione {Benzofuran-4,5-dione} was synthesized using p-benzoquinone and 1-(N-morpholine)propene. 1.5 g (9.3 mM) of the thus-prepared benzofuran-4,5-dione and 3.15 g (28.2 mM) of 1-acetoxy-1,3-butadiene were dissolved in 200 ml of benzene, and the resulting mixture was refluxed for 12 hours. The reaction solution was cooled to room temperature and concentrated by distillation under reduced pressure. This was followed by chromatography on silica gel to give 1.13 g of pure Compound 30.

¹H-NMR (CDCl₃, δ): 8.05 (1H, dd, J=1.2, 7.6 Hz), 7.68 (1H, dd, J=1.2, 7.6 Hz), 7.64 (1H, td, J=1.2, 7.6 Hz), 7.43 (1H, td, J=1.2, 7.6 Hz), 7.26 (1H, q, J=1.2 Hz), 2.28 (3H, d, J=1.2 Hz)

### Example 29

### Synthesis of Compounds 31 and 32

1.5 g (9.3 mM) of 4,5-dihydro-3-methylbenzo[1,2-b]furan-4,5-dione {Benzofuran-4,5-dione} and 45 g (0.6M) of 2-methyl-1,3-butadiene were dissolved in 200 ml of benzene, and the resulting mixture was refluxed for 5 hours. The reaction solution was cooled to room temperature and completely concentrated by distillation under reduced pressure. The thus-obtained concentrates were dissolved again in 150 ml of carbon tetrachloride, followed by addition of 2.3 g (10 mM) of 2,3-dichloro-5,6-dicyano-1,4-benzoqinone, and the resulting mixture was further refluxed for 15 hours. The reaction solution was cooled and concentrated by distillation under reduced pressure. The resulting concentrates were purified by chromatography on silica gel to give 0.13 g and 0.11 g of pure Compounds 31 and 32, respectively.

¹H-NMR (CDCl₃, δ) of Compound 31: 7.86 (1H, s), 7.57 (1H, d, J=8.1 Hz), 7.42 (1H, d, J=8.1 Hz), 7.21 (1H, q, J=1.2 Hz), 2.40 (3H, s), 2.28 (1H, d, J=1.2 Hz)

¹H-NMR (CDCl₃, δ) of Compound 32: 87.96 (1H, d, J=8.0 Hz), 7.48 (1H, s), 7.23 (2H, m), 2.46 (3H, s), 2.28 (1H, d, J=1.2 Hz)

### Experimental Example 1

### Effects of AMPK Activation on Relaxation of Penile Corpus Cavernosum Smooth Muscles

### a) Preparation of Penile Corpus Cavernosum Section and Measurement of Relaxation Effects Thereof

A New Zealand white rabbit (2.5 to 3.0 kg) was anesthetized and the overall penis was then dissected from the rabbit. The penile corpus cavernosum smooth muscles were separated from the tunica albuginea in a low-temperature Tyrode's solution aerated with a 95% O₂-5% CO₂ gas mixture, while observing with a dissection microscope, to prepare a penis section. The penis section was fixed in an organ bath (10 cc) containing a Tyrode's solution. At this time, the one end of the penis section was fixed on the bottom of the organ bath, and a variation in isometric tension of the penile corpus cavernosum smooth muscles was then measured with a force displacement transducer at the other end thereof. After reaching an equilibrium state, the penile section was contracted by treatment of 10⁻⁴ M phenylephrine. The penile section was treated with gradually increased concentrations (10⁻⁹ to 10⁻⁴ M) of AMPK activators and comparative drugs, and the effects on relaxation of the penile corpus cavernosum were then observed.

### b) Results of Relaxation Effect Measurements

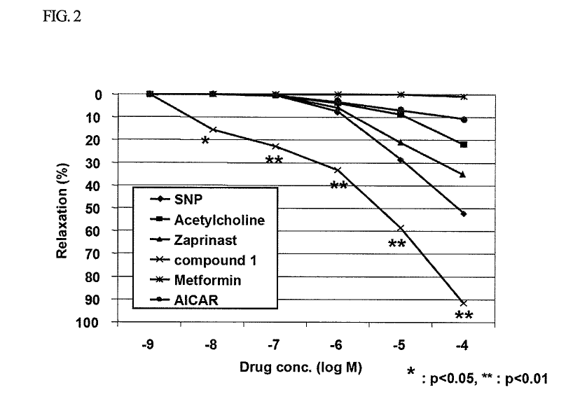

According to the drug used to treat the penile section, the experimental groups used herein were divided into six groups, i.e., i) a group with administration of SNP as a NO donor, ii) a group with administration of Zaprinast as an inhibitor against PDE-5, an enzyme that mediates hydrolysis of c-GMP, a group with administration of acetylcholine that acts on the nervous system, iv) a group with administration of metformin, one of conventional AMPK activators, v) a group with administration of AICAR (5-aminoimidazole-4-carboxamide-1-beta-D-ribofuranoside), and vi) a group with administration of the compound 1 according to the present invention. These groups were compared with one another, in view of relaxation (%) of the penile corpus cavernosum. Table 2 shows relaxation (%) of penile corpus cavernosum smooth muscles of the groups, to which the corresponding compound is administered with a constant concentration of 10⁻⁴ M. FIG. 2 shows relaxation (%) of the penile corpus cavernosum smooth muscles of the groups, to which the corresponding compound is administered with a gradually increased concentration 10⁻⁹ to 10⁻⁴ M).

TABLE 2

| Administered Compound (conc. 10⁻⁴ M) | Relaxation (%) |
|---|---|
| SNP | 53 |
| Zaprinast | 36 |
| Acetylcholine | 21 |
| Metformin | 0.1 |
| AICAR | 10 |
| Example 1 (compound 1) | 92 |

As apparent from the data shown in Table 2, in a case where the administration concentration is 10⁻⁴ M, the relaxation of the penile corpus cavernosum smooth muscles is in the order of metformin<AICAR<acetylcholine<zaprinast<SNP<the compound 1. From FIG. 2, it can be confirmed that in a concentration range of 10⁻⁹ to 10⁻⁷ M, all of the compounds except the compound 1 according to the present invention hardly exert relaxation effects, but the compound 1-administration group exhibits a very high relaxation (%) that corresponds to about double that of the SNP-administration group and exerts superior relaxation effects throughout the range of 10⁻⁹ to 10⁻⁴ M.

As can be seen from the foregoing, the pharmaceutical composition according to the present invention exhibits considerably excellent effects on relaxation of penile corpus cavernosum smooth muscles, as compared to active ingredients of conventional pharmaceutical compositions and AMPK activators, and furthermore exerts potent efficacies despite the administration of a relatively small dosage. Accordingly, these results demonstrate that the pharmaceutical composition of the present invention is suitable for use as a novel medicine for erectile dysfunction.

### Experimental Example 2

### Effects of the Compound on Phosphorylation of eNOS

In order to ascertain whether the compound 1 acting as an AMPK activator is implicated in the production of NO, phosphorylation to promote the activity of endothelial nitric oxide synthase (eNOS) was measured. So as to confirm the phosphorylation of eNOS through the compound 1, HUVEC cells were seeded on a 60 mm plate at 1×10⁵ in an EBM2/5% FBS medium and cultured for 24 hours. The medium was replaced by a serum-free EBM2 medium and the treatment with the compound 1 (10 uM) was performed for a predetermined period. An anti-pS1177 eNOS was used to measure the phosphorylated eNOS.

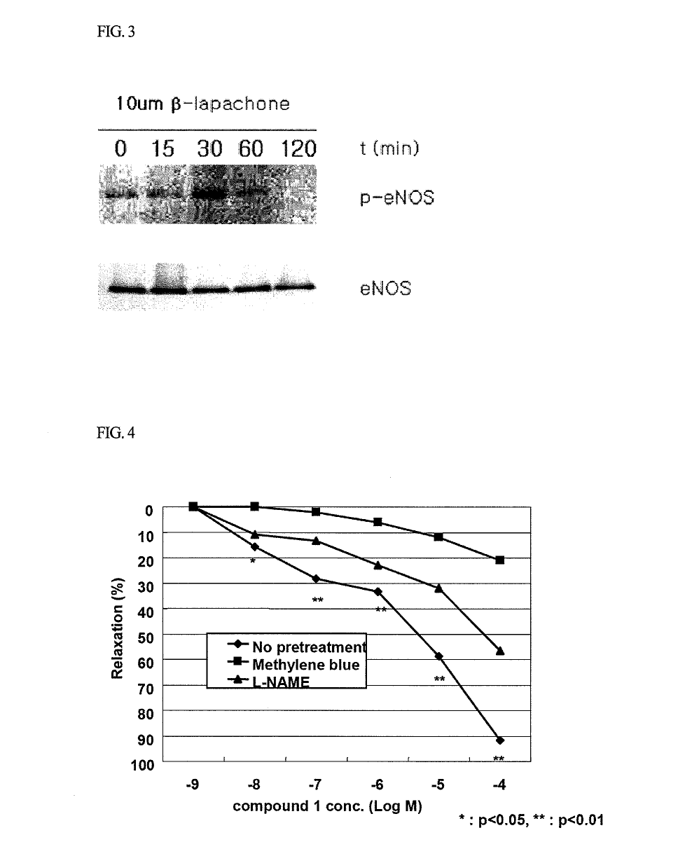

As can be seen from FIG. 3, phosphorylation of eNOS (p-eNOS) is maximized at 30 minutes following the treatment with the compound 1, then is gradually reduced and is not observed at 2 hours, and the activity of eNOS is observed all through the 2 hours. Thus, these results indicate that the compound 1 induces the activation of AMPK in endothelial cells, resulting in the phosphorylation and activity of eNOS and thus at least partially promoting NO production.

### Experimental Example 3

### Effects of Compound 1 on Relaxation Through NO Pathway

To confirm the effects of the compound 1 on relaxation of the penile corpus cavernosum smooth muscles upon blocking of a NO pathway, the penile corpus cavernosum smooth muscle sections were contracted by the treatment of phenylephrine, relaxed by the treatment of the compound 1, and then sequentially treated with methylene blue as an inhibitor against guanylate cyclase that helps production of c-GMP and with L-NAME (L-nitroarginine methyl ester) that inhibits production of NO via inhibitory activity against eNOS.

The relaxation of the penile corpus cavernosum smooth muscles caused by the administration of the compound 1 were partially inhibited by 10⁻³ M of L-NAME and 10⁻³ M of methylene blue. The results are shown in FIG. 4.

As can be seen from FIG. 4, the relaxation of L-NAME (10⁻³ M)-treatment group was inhibited, as compared to a no-treatment group a group with administration of the compound 1). This behavior indicates that the compound 1 according to the present invention activates eNOS, enhancing production of NO and inducing relaxation of penile corpus cavernosum smooth muscles.

In addition, as can be seen from FIG. 4, the relaxation of a group with treatment of methylene blue 10⁻⁴ M was more greatly inhibited, as compared to the L-NAME-treatment group. This behavior ascertains that the compound 1 of the present invention induces relaxation of the penile corpus cavernosum smooth muscles by promoting the production of cGMP, mainly through the NO-cGMP pathway.

In conclusion, these results demonstrate that the compound 1 is implicated in endothelium-dependent NO production pathway and NO-cGMP pathway via promotion of eNOS activation and cGMP production, to induce relaxation of the penile corpus cavernosum smooth muscles.

### Experimental Example 4

### Effects of the Compound 1 on Relaxation Through Co-Production Pathway

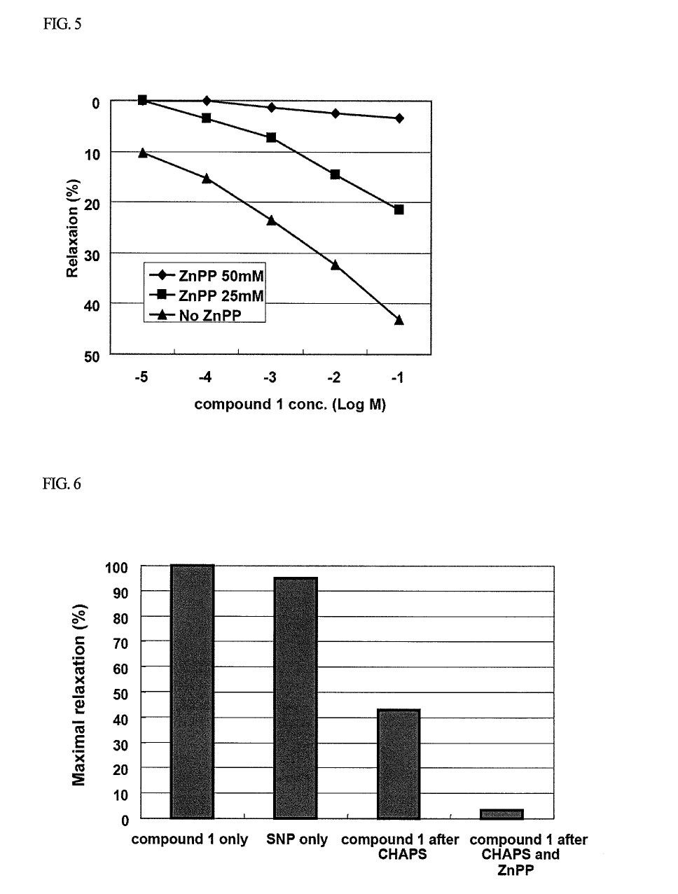

To ascertain the pharmacological mechanism the compound 1 acts on the relaxation of the penile corpus cavernosum smooth muscles in the New Zealand white rabbits, the smooth muscle sections were relaxed by the treatment of the compound 1 and endothelial cells were then removed from the smooth muscle sections using 10⁻⁴ M of CHAPS (3-[(3-cholamidopropyl) dimethylammonio]-1-propanesulfonate, 0.3% in buffer solution; Sigma) as a lysis buffer. Subsequently, as another neurotransmitter, zinc-protoporphyrin-IX (ZnPP) was administrated thereto, which inactivates heme oxygenase-2 (HO-2) that produces carbon monoxide (CO) that is in vivo produced by blood and acts as a vasodilator. The results are shown in FIGS. 5 and 6.

From FIGS. 5 and 6, it can be confirmed that the compound 1-administration group exhibited significantly excellent relaxation effects, as compared to the SNP-administration group, and inhibition of the only NO pathway cannot completely inactivate the relaxation effects. That is to say, it was not until both the NO pathway and the CO pathway are inhibited that relaxation effects can be almost completely suppressed. In addition, it can be confirmed that the relaxation of the CHAPS-administration group (no Znpp) was decreased to about 45% of the relaxation of the compound 1-administration group (not shown), and that relaxation of the penile corpus cavernosum smooth muscles of the CHAPS-administration group was partially inhibited. Accordingly, these results indicate that the compound 1 according to the present invention also exhibits endothelium-independent activities. The relaxation of the ZnPP-administration group was almost completely inhibited. This ascertains that the compound 1 is implicated in an endothelium-independent CO-production pathway which suppresses production of CO acting as a vasodilator via activation of HO-2.

In conclusion, when the compound 1 is treated and only the endothelium-dependent NO-production pathway is blocked, the relaxation of the penile corpus cavernosum smooth muscles is partially inhibited. Meanwhile, when the endothelium-independent CO-production pathway is blocked, the relaxation is completely inhibited. These behaviors demonstrate that the compound 1 induces relaxation of the penile corpus cavernosum smooth muscles through the pharmacological mechanism which is implicated in both the endothelium-dependent NO-production pathway and the endothelium-independent CO-production pathway.

### Experimental Example 5

### Influence of the Compound 1 on Internal Pressure of Erected Penile Corpus Cavernosum of Diabetes-Induced Rats

In order to confirm in vivo whether penile erection of a diabetes-induced rat has an influence on the internal pressure of the penile corpus cavernosum, 23 Sprague-Dawley white rats (about 300 g) are divided into a normal control group (Group I, n=6), a diabetes-induced control group (Group II, n=4), a group with administration of AICAR following the diabetes-induction (Group III, n=6), and a group with administration of the compound 1 (Group IV, n=7).

STZ (streptozotocin) was used to induce diabetes, and the AICAR and the compound 1 were orally administered in dosages of 500 mg/kg and 250 mg/kg, respectively, for 5 weeks. Each of the experimental groups was systemically anesthetized, intubated into one-side of the carotid in order to measure blood pressure, a electrostimulation-purpose catheter was set in the penile corpus cavernosum nerves in the cavum pelvis, and the internal pressure of the penile corpus cavernosum was measured. Electrostimulation was applied to the group for one minute (frequency: 10 Hz, delay: 4 ms, duration: 5 ms, volt: 3V). An increase in internal pressure of the groups was measured, and the groups were compared with one another in view of the increase in internal pressure.

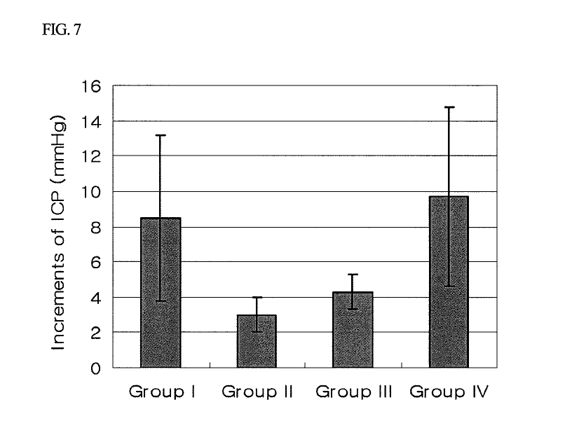

The increase in internal pressure is shown in FIG. 7. There was neither significant difference in the internal pressure of the penile corpus cavernosum nerves measured prior to the electrostimulation between the respective groups, nor variation in systemic blood pressure during the electrostimulation.

It can be seen from FIG. 7 that averages of the highest internal pressure increase of the penile corpus cavernosum after the electrostimulation with respect to the groups I, II, III and IV were 8.5±4.7, 3.0±1.0, 4.3±1.0, and 9.7±5.1 mmHg, respectively. The average of the Group IV with administration of the compound 1 is significantly higher than that of Group II or III (p=0.022). In view of the internal pressure increase of the penile corpus cavernosum, the group IV showed no statistically significant difference with the Group I (p=0.670), but is statistically significantly higher than the Group III (p=0.027).

Accordingly, there results demonstrate that the compound 1 significantly elevates the internal pressure of the penile corpus cavernosum of diabetes-induced white rats. As a result, the pharmaceutical composition comprising the compound 1 as an active ingredient is expected to be a novel medicine for treating erectile dysfunction including diabetes-related erectile dysfunction.

### INDUSTRIAL APPLICABILITY

As apparent from the above description, the pharmaceutical composition of the present invention is implicated in an endothelium-dependent NO pathway and a endothelium-independent HO-2 pathway via activation of AMPK, promoting production of NO and CO that act as neurotransmitters, and thus being significantly effective for inducing relaxation of penile corpus cavernosum smooth muscles despite use of a relatively small dosage. Accordingly, the pharmaceutical composition of the present invention is preferably useful for treatment and prevention of erectile dysfunction.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A method of treating and/or preventing erectile dysfunction, comprising:
administering to a subject in need thereof a composition, comprising:
(a) a therapeutically effective amount of one or more selected from the compounds represented by Formula 1 and Formula 2 below: 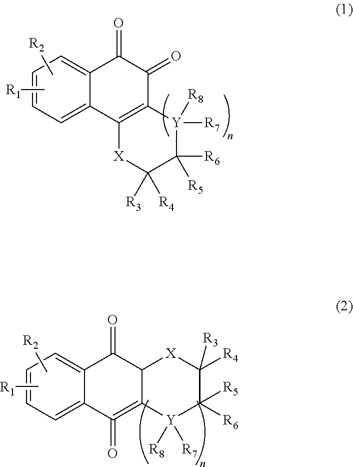
wherein,
R₁ and R₂ are each independently hydrogen, halogen, hydroxyl, or C₁-C₆ lower alkyl or alkoxy, or R₁ and R₂ may be taken together to form a substituted or unsubstituted cyclic structure which may be saturated or partially or completely unsaturated;
R₃, R₄, R₅, R₆, R₇ and R₈ are each independently hydrogen, hydroxyl, C₁-C₂₀ alkyl, alkene or alkoxy, or C₄-C₂₀ cycloalkyl, heterocycloalkyl, aryl or heteroaryl, or two substituents of R₃ to R₈ may be taken together to form a cyclic structure which may be saturated or partially or completely unsaturated;
X is selected from the group consisting of C(R)(R′), N(R″), O and S, wherein R, R′ and R″ are each independently hydrogen or C₁-C₆ lower alkyl;
Y is C, S or N, with proviso that when Y is S, R₇ and R₈ are not any substituent, and when Y is N, R₇ is hydrogen or C₁-C₆ lower alkyl and R₈ is not any substituent; and
n is 0 or 1, with proviso that when n is 0, carbon atoms adjacent ton forma cyclic structure via a direct bond; or a pharmaceutically acceptable salt, prodrug, solvate or isomer thereof; and
(b) a pharmaceutically acceptable carrier, a diluent or an excipient, or any combination thereof.

2. The method according to claim 1, wherein X is O.

3. The method according to claim 1, wherein the prodrug is a compound represented by Formula 1a below: 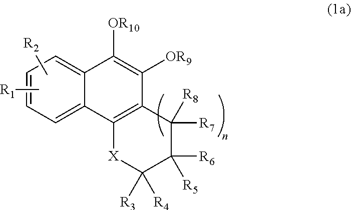
wherein,
R₁, R₂, R₃, R₄, R₅, R₆, R₇, R₈, X and n are as defined in Formula 1;
R₉ and R₁₀ are each independently —SO₃—Na⁺ or substituent represented by Formula A below or a salt thereof, 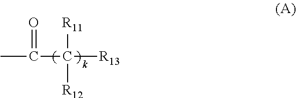
wherein,
R₁₁ and R₁₂ are each independently hydrogen or substituted or unsubstituted C₁-C₂₀ linear alkyl or C₁-C₂₀ branched alkyl,
R₁₃ is selected from the group consisting of substituents i) to viii) below,
i) hydrogen;
ii) substituted or unsubstituted C₁-C₂₀ linear alkyl or C₁-C₂₀ branched alkyl;
iii) substituted or unsubstituted amine;
iv) substituted or unsubstituted C₃-C₁₀ cycloalkyl or C₃-C₁₀ heterocycloalkyl;
v) substituted or unsubstituted C₄-C₁₀ aryl or C₄-C₁₀ heteroaryl;
vi) —(CRR′—NR″CO)₁—R₁₄, wherein R, R′ and R″ are each independently hydrogen or substituted or unsubstituted C₁-C₂₀ linear alkyl or C₁-C₂₀ branched alkyl, R₁₄ is selected from the group consisting of hydrogen, substituted or unsubstituted amine, cycloalkyl, heterocycloalkyl, aryl and heteroaryl, 1 is selected from the 1˜5;
vii) substituted or unsubstituted carboxyl;
viii) —OSO₃—Na⁺;
k is selected from the 0˜20, with proviso that when k is 0, R₁₁ and R₁₂ are not anything, and R₁₃ is directly bond to a carbonyl group.

4. The method according to claim 1, wherein the compound of Formula 1 is selected from compounds of Formulas 3 and 4 below: 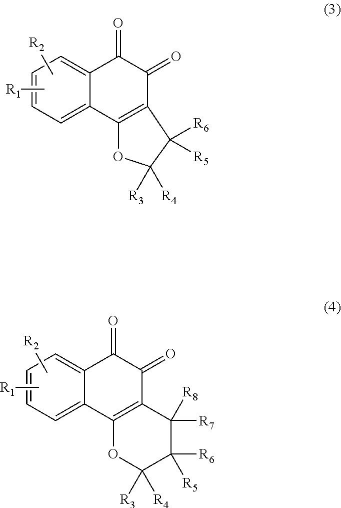
wherein R₁, R₂, R₃, R₄, R₅, R₆, R₇ and R₈ are as defined in Formula 1.

5. The method according to claim 1, wherein each of R₁ and R₂ is respectively hydrogen.

6. The method according to claim 4, wherein the compound of Formula 3 is a compound of Formula 3a below in which R₁, R₂ and R₄ are respectively hydrogen, or a compound of Formula 3b below in which R₁, R₂ and R₆ are respectively hydrogen:

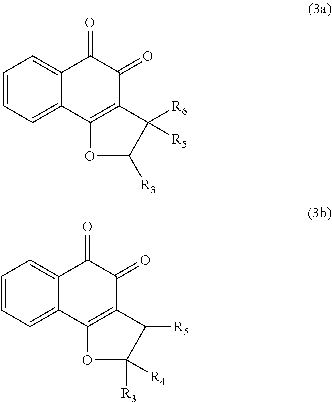

7. The method according to claim 4, wherein the compound of Formula 4 is selected from compounds of Formulas 4a to 4c below:

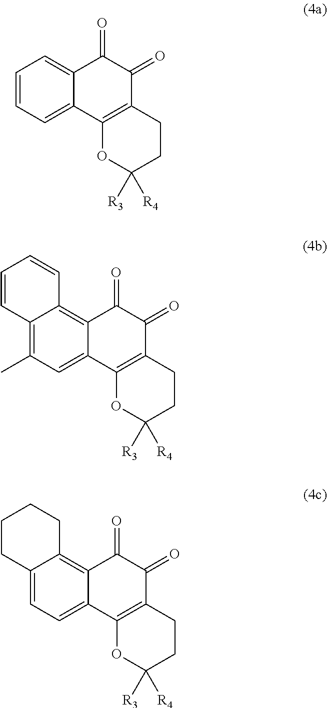

8. The method according to claim 1, wherein the compound of Formula 2 is a compound of Formula 2a in which n is 0 and adjacent carbon atoms form a cyclic structure via a direct bond therebetween and Y is C, or a compound of Formula 2b in which n is 1 Y is C: 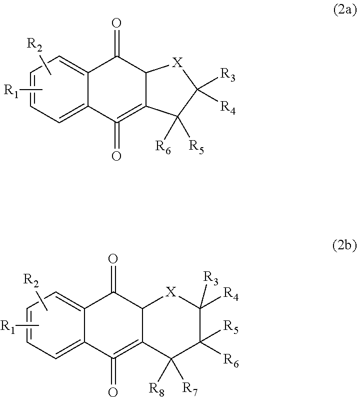
wherein R₁, R₂, R₃, R₄, R₅, R₆, R₇, R₈ and X are as defined in Formula 1.

9. The method according to claim 1, wherein the compound of Formula 1 or Formula 2 is contained in a crystalline structure.

10. The method according to claim 1, wherein the compound of Formula 1 or Formula 2 is contained in an amorphous structure.

11. The method according to claim 1, wherein the compound of Formula 1 or Formula 2 is formulated into the form of a fine particle.

12. The method according to claim 11, wherein the formulation for form of a fine particle is carried out by using the particle micronization method selected from the group consisting of mechanical milling, spray drying, precipitation method, homogenization, and supercritical micronization.

13. The method according to claim 12, wherein the formulation is carried out by using jet milling as a mechanical milling and/or spray drying.

14. The method according to claim 11, wherein the particle size of fine particles is 5 nm to 500 μm.

15. The method according to claim 1, wherein the pharmaceutical composition is prepared into an intestine-targeted formulation.

16. The method according to claim 15, wherein the intestine-targeted formulation is carried out by addition of a pH sensitive polymer.

17. The method according to claim 15, wherein the intestine-targeted formulation is carried out by addition of a biodegradable polymer which is decomposable by an intestine-specific bacterial enzyme.

18. The method according to claim 15, wherein the intestine-targeted formulation is carried out by addition of a biodegradable matrix which is decomposable by an intestine-specific bacterial enzyme.

19. The method according to claim 15, wherein the intestine-targeted formulation is carried out by a configuration with time-course release of the drug after a lag time (‘time-specific delayed-release formulation’).

20. The method according to claim 1, wherein the erectile dysfunction is diabetes-related erectile dysfunction.
